SEQUENCE LISTING (8 sequences)

SEQ ID NO: 1 - protein, 805 aa, Artificial Sequence [sequence omitted]

SEQ ID NO: 2 - DNA, 2415 bp, Artificial Sequence
  Synthetic
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttt

SEQ ID NO: 3 - RNA, 19 nt, Artificial Sequence
  Synthetic
acaugaggaucacccaugu

SEQ ID NO: 4 - protein, 117 aa, Artificial Sequence
  Synthetic
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 5 - RNA, 25 nt, Artificial Sequence
  Synthetic
ggagcagacgauauggcgucgcucc

SEQ ID NO: 6 - protein, 123 aa, Artificial Sequence
  Synthetic
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 7 - RNA, 15 nt, Artificial Sequence
  Synthetic
gcccugaagaagggc

SEQ ID NO: 8 - protein, 23 aa, Artificial Sequence
  Synthetic
MGNARTRRRERRAEKQAQWKAAN

(19) United States
(12) Patent Application Publication
(10) Pub. No.: US 2023/0399641 A1
(43) Pub. Date: Dec. 14, 2023

(54) GENOMIC EDITING OF IMPROVED EFFICIENCY AND ACCURACY

(71) Applicant: SHANGHAITECH UNIVERSITY, Shanghai (CN)

(72) Inventors: Jia CHEN, Shanghai (CN); Bei YANG, Shanghai (CN); Li YANG, Shanghai (CN); Xiaosa LI, Shanghai (CN); Xiao WANG, Shanghai (CN); Wenwen ZHAO, Shanghai (CN); Lina ZHOU, Shanghai (CN); Jifang LI, Shanghai (CN)

(21) Appl. No.: 18/036,399

(22) PCT Filed: Nov. 11, 2021
(86) PCT No.: PCT/CN2021/130059

§ 371 Date: May 10, 2023

(30) Foreign Application Priority Data
Nov. 12, 2020 (WO) PCT/CN2020/128417
May 6, 2021 (WO) PCT/CN2021/091939

(51) Int. Cl. C12N 15/11; C12N 9/22; C12N 9/12; C12N 15/90

(52) U.S. Cl. CPC C12N 15/111; C12N 9/22; C12N 9/1276; C12Y 207/07049; C12N 15/907; C12N 2310/20; C07K 2319/00

(57) ABSTRACT

Provided are compositions and methods for enhanced prime editing, which include a pegRNA that encodes a target mutation in a target protein, along with one or more nearby silent or conservative mutations. These silent mutations can increase the editing efficiency, without causing a change to the target protein sequence. Also provided are compositions and methods of using the improved prime editing for preventing or treating infections by SARS-CoV or SARS-CoV-2.

### BACKGROUND

Genome editing is a new form of genetic engineering in which DNA is inserted, deleted or replaced in the genome of a living organism using engineered nucleases (molecular scissors). Utilizing genome editing tools to genetically manipulate the genome of cells and living organism has broad interest in life science research, biotechnology, agricultural technology and most importantly disease treatment. For example, genome editing could be used to correct the driver mutations causing genetic diseases, thereby resulting in cure of these diseases in living organism; genome editing could also be applied to engineer the genome of crops, thus increasing the yield of crops and conferring crops resistance to environmental contamination or pathogen infection; likewise, microbial genome transformation through accurate genome editing is of great significance in the development of renewable bio-energy.

The CRISPR/Cas (Clustered regularly interspaced short palindromic repeats/CRISPR-associated protein) system has been the most powerful genomic editing tool since its conception for its high editing efficiency, convenience and the potential applications in living organism. Directed by a guide RNA (gRNA), the Cas nuclease can generate DNA double strand breaks (DSBs) at the targeted genomic sites in various cells (both cell lines and primary cells from living organisms). These DSBs are then repaired by endogenous DNA repair systems, which could be utilized to perform desired genome editing.

In general, two major DNA repair pathways could be activated by DSBs, non-homologous end joining (NHEJ) and homology-directed repair (HDR). NHEJ could introduce random insertions/deletions (indels) in the genomic DNA region around the DSBs, thereby leading to open reading frame (ORF) shift and ultimately gene inactivation. In contrast, when HDR is triggered, the genomic DNA sequence at target site could be replaced by the sequence of the exogenous donor DNA template through a homologous recombination mechanism, which can result in the correction of genetic mutation. However, the practical efficiency of HDR-mediated gene correction is low (normally less than 5%) because the occurrence of homologous recombination is both cell type-specific and cell cycle-dependent and NHEJ is triggered more frequently than HDR is. The relatively low efficiency of HDR therefore limited the translation of CRISPR/Cas genome editing tools in the field of therapies involving gene correction.

Prime editor (PE), which integrates the CRISPR/Cas system with the reverse transcriptase (RTase) family, was recently invented for gene correction. By fusing to Cas9 nickase (nCas9, H840A), RTases can mediate reverse transcription at the target genomic locus by using the genetic information encoded in a prime editing gRNA (pegRNA) and then trigger the incorporation of complementary DNA (cDNA) into genomic DNA, which will eventually lead to intended editing.

However, compared to those of other genome editors, the editing efficiency of PE is reactively low, particularly when generating single base changes. Thus, developing new prime editing systems with high editing efficiencies is desirable. Such prime editing systems will enable us to perform high levels editing in various living organisms. Importantly, the high efficiency of these new prime editing systems will promote potential clinical translations, particularly in those involve correcting disease-related single point mutations.

### SUMMARY

It is discovered herein that, by introducing one or more silent mutations (mutations in the coding sequence that does not cause a change of amino acid or mutations affect only noncoding DNA) to the prime editing gRNA (pegRNA), the editing efficiency of prime editing can be greatly enhanced. The endogenous mismatch repair (MMR) system employed by the prime editing system is not efficient in repairing single-base mismatches. The silent mutations introduced in the pegRNAs, it is contemplated, can cause more mismatches and larger distortion of DNA structure, leading to enhanced MMR activation and prime editing.

In accordance with one embodiment of the present disclosure, therefore, provided is a method for generating a target mutation to a protein in a cell. In some embodiments, the method comprises introducing to the cell a prime editing system, wherein the prime editing system comprises a fusion protein comprising a nickase and a reverse transcriptase, and a prime editing guide RNA (pegRNA) encoding the target mutation and one or more silent or conservative mutations within 20 nucleotides from the target mutation.

In another embodiment, provided is a method for introducing mutations to a protein in a cell, comprising introducing to the cell a prime editing system, wherein the prime editing system comprises a fusion protein comprising a nickase and a reverse transcriptase, and a prime editing guide RNA (pegRNA) encoding two or more mutations within the coding sequence of the protein, wherein at least one of the mutations is a silent or conservative mutation and is within 20 nucleotides from another of mutations.

Also provided, in one embodiment, is a prime editing guide RNA (pegRNA) comprising a fragment that (a) is capable of hybridizing to the genomic sequence of human ACE2 (angiotensin-converting enzyme 2) and (b) encodes a target mutation at one or more residues selected from the group consisting of S19, Q24, D30, K31 or K353, which positions are according to SEQ ID NO: 1. Also provided are such mutant proteins, polynucleotides encoding the mutant proteins, cells that contain the mutant proteins, and antibodies that specifically recognize the mutant proteins.

Also provided is a prime editing system, comprising the pegRNA of the present disclosure and a fusion protein comprising a nickase and a reverse transcriptase.

Methods for treating or preventing infection by a coronavirus are also provided. In some embodiments, the methods comprise administering to a subject one or more polynucleotides encoding the prime editing system of the disclosure. In some embodiments, the coronavirus is SARS-CoV-2 (severe acute respiratory syndrome coronavirus 2) or SARS-CoV.

Also provided, in some embodiments, is a “split PE” system for conducting genetic editing. In one embodiment, provided is a method for conducting genetic editing in a cell at a target site, comprising introducing to the cell a first construct, which can be enclosed in a first viral particle, encoding a nickase, and a second or more construct, which can be enclosed in a second viral particle, encoding (a) a prime editing guide RNA (pegRNA) capable of identifying the target site and including genetic information for editing the target site, (b) a single guide RNA (sgRNA) capable of directing the nickase to nick a non-edited DNA strand of the target site, wherein the pegRNA or the sgRNA includes a tag sequence, and (c) a reverse-transcriptase fused to an RNA recognition peptide capable of binding to the tag sequence.

### BRIEF DESCRIPTION OF THE DRAWINGS

FIG. 1. pegRNAs encoding silent mutations induced efficient pathogenic T89I mutation in ACTG1 gene. 1A: Schematic diagram illustrating the original and silent mutation-encoding pegRNAs for inducing T89I mutation in ACTG1 gene. 1B: Schematic diagram illustrating the co-transfection of the plasmids expressing PE2, sgACTG1-nicking and pegRNAs for inducing T89I mutation in ACTG1 gene. 1C: Comparison of T89I mutation induced by the original and silent mutation-encoding pegRNAs. Solid box represents the position of pathogenic T89I mutation. Dashed boxes represent the positions of induced silent mutations.

FIG. 2. pegRNAs encoding silent mutations induced efficient pathogenic L23M mutation in CFTR gene. 2A: Schematic diagram illustrating the original and silent mutation-encoding pegRNAs for inducing L23M mutation in CFTR gene. 2B: Schematic diagram illustrating the co-transfection of the plasmids expressing PE2, sgCFTR-nicking and pegRNAs for inducing L23M mutation in CFTR gene. 2C: Comparison of L23M mutation induced by the original and silent mutation-encoding pegRNAs. Solid box represents the position of pathogenic L23M mutation. Dashed boxes represent the positions of induced silent mutations.

FIG. 3. pegRNAs encoding silent mutations induced efficient pathogenic C160G mutation in FBN1 gene. 3A: Schematic diagram illustrating the original and silent mutation-encoding pegRNAs for inducing C160G mutation in FBN1 gene. 3B: Schematic diagram illustrating the co-transfection of the plasmids expressing PE2, sgFBN1-nicking and pegRNAs for inducing C160G mutation in FBN1 gene. 3C: Comparison of C160G mutation induced by the original and silent mutation-encoding pegRNAs. Solid box represents the position of pathogenic C160G mutation. Dashed boxes represent the positions of induced silent mutations.

FIG. 4. pegRNAs encoding silent mutations induced efficient pathogenic K18Ter mutation in HBB gene. 4A: Schematic diagram illustrating the original and silent mutation-encoding pegRNAs for inducing K18Ter mutation in HBB gene. 4B: Schematic diagram illustrating the co-transfection of the plasmids expressing PE2, sgHBB-nicking and pegRNAs for inducing K18Ter mutation in HBB gene. 4C: Comparison of K18Ter mutation induced by the original and silent mutation-encoding pegRNAs. Solid box represents the position of pathogenic K18Ter mutation. Dashed boxes represent the positions of induced silent mutations.

FIG. 5. The interface between human ACE2 and the RBD domain of spike proteins from SARS-CoV and SARS-CoV-2. 5A: The interface between human ACE2 and the RBD domain from SARS-CoV-2 spike. 5B: The interface between human ACE2 and the RBD domain from SARS-CoV spike.

FIG. 6. The pair of pegACE2-S19A|sgACE2-nicking can efficiently induce S19A mutation. 6A: Schematic diagram illustrating pegACE2-S19A. 6B: Schematic diagram illustrating the co-transfection of the plasmids expressing PE2, sgACE2-nicking and pegACE2-S19A. 6C: The pair of pegACE2-S19A|sgACE2-nicking can efficiently induce S19A (T55C base substitution in the coding region of ACE2 gene) mutation. Dashed boxes represent the location of T55C base editing at pegACE2-S19A target site.

FIG. 7. The pair of pegACE2-Q24N|sgACE2-nicking and pegACE2-Q24del|sgACE2-nicking can efficiently induce Q24N and Q24del mutations. 7A: Schematic diagram illustrating pegACE2-Q24N and pegACE2-Q24del. 7B: Schematic diagram illustrating the co-transfection of the plasmids expressing PE2, sgACE2-nicking and the plasmid expressing pegACE2-Q24N or pegACE2-Q24del. 7C: The pair of pegACE2-Q24N|sgACE2-nicking and pegACE2-Q24del|sgACE2-nicking can efficiently induce Q24N (C70A/G72C base substitutions in the coding region of ACE2 gene) and Q24del (C70Del/A71Del/G72Del base deletions in the coding region of ACE2 gene) mutations. Dashed boxes represent the locations of base editing at pegACE2-Q24N and pegACE2-Q24del target site.

FIG. 8. The pair of sgACE2-nicking and pegACE2 encoding double or triple amino-acid mutations can efficiently induce intended base substitutions in the coding region of ACE2 gene. 8A: Schematic diagram illustrating pegACE2 encoding double or triple amino-acid mutations (pegACE2-Q24A/D30A, pegACE2-Q24A/K31A, pegACE2-Q24A/D30A/K31A and pegACE2-Q24A/D30K/K31A). 8B: Schematic diagram illustrating the co-transfection of the plasmids expressing PE2, sgACE2-nicking and the plasmids expressing pegACE2-Q24A/D30A, pegACE2-Q24A/K31A, pegACE2-Q24A/D30A/K31A or pegACE2-Q24A/D30K/K31A. 8C: The pair of pegACE2-Q24A/D30A|sgACE2-nicking, pegACE2-Q24A/K31A|sgACE2-nicking, pegACE2-Q24A/D30A/K31A|sgACE2-nicking and pegACE2-Q24A/D30K/K31A|sgACE2-nicking can efficiently induce Q24A/D30A (C70G/A71C/G72C/A89C base substitutions in the coding region of ACE2 gene), Q24A/K31A (C70G/A71C/G72C/A91G/A92C/G93C base substitutions in the coding region of ACE2 gene), Q24A/D30A/K31A (C70G/A71C/G72C/A89C/A91G/A92C/G93C base substitutions in the coding region of ACE2 gene) and Q24A/D30K/K31A (C70G/A71C/G72C/G88A/C90G/A91G/A92C/G93C base substitutions in the coding region of ACE2 gene) mutations. Dashed boxes represent the locations of base editing at pegACE2 target sites.

FIG. 9. The pair of sgACE2-nicking and pegACE2 encoding quadruple amino-acid mutations can efficiently induce intended base substitutions in the coding region of ACE2 gene. 9A: Schematic diagram illustrating pegACE2-Q24A/D30A/K31A/D38A and pegACE2-Q24A/D30K/K31A/D38A. 9B: Schematic diagram illustrating the co-transfection of the plasmids expressing PE2, sgACE2-nicking and the plasmid expressing pegACE2-Q24A/D30A/K31A/D38A or pegACE2-Q24A/D30K/K31A/D38A. 9C: The pair of pegACE2-Q24A/D30A/K31A/D38A|sgACE2-nicking and pegACE2-Q24A/D30K/K31A/D38A|sgACE2-nicking can efficiently induce Q24A/D30A/K31A/D38A (C70G/A71C/G72C/A89C/A91G/A92C/G93C/A113C base substitutions in the coding region of ACE2 gene) and Q24A/D30K/K31A/D38A (C70G/A71C/G72C/G88A/C90G/A91G/A92C/G93C/A113C base substitutions in the coding region of ACE2 gene) mutations. Dashed boxes represent the locations of base editing at pegACE2 target sites.

FIG. 10. The RBD-binding affinity and enzymatic activity of wild-type and engineered human ACE2. 10A: The Kₒₙ, K_{off} and disassociation constant (K_{D}) between ACE2 mutants and the RBD domain from SARS-CoV-2 spike were measured by SPR, and the relative affinity of mutants were normalized against that of wildtype ACE2. 10B: The angiotensin-converting activity of wildtype and engineered ACE2. The Vₘₐₓ of mutants were normalized against that of wildtype ACE2. *, P<0.05; N.S., not significant (Student's t test).

FIG. 11 illustrates a split PE3 system (panel B) in comparison with the original PE3 system (panel A).

FIG. 12 shows comparison of the editing efficiencies of PE3 and split PE3 for inducing Q24del mutation in the coding region of ACE2 gene. 12A: Schematic diagram illustrating the constructs for PE3 and split PE3 when mediating Q24del mutation in the coding region of ACE2 gene. 12B: Similar editing efficiencies by PE3 and split PE3 when mediating Q24del mutation (C70Del/A71Del/G72Del base deletions in the coding region of ACE2 gene).

FIG. 13 shows comparison of the editing efficiencies of PE3 and split PE3 for inducing Q24A/D30A/K31A mutation in the coding region of ACE2 gene. 13A: Schematic diagram illustrating the constructs for PE3 and split PE3 when mediating Q24A/D30A/K31A mutation in the coding region of ACE2 gene. 13B: Similar editing efficiencies by PE3 and split PE3 when mediating Q24A/D30A/K31A mutation (C70G/A71C/G72C/A89C/A91G/A92C/G93C base substitutions in the coding region of ACE2 gene).

FIG. 14. pegRNA containing additional base substitutions induced higher editing efficiency of single base substitution. (a) Sequences of primer-binding site (PBS) and RT-template (RTT) of pegRNAs and wild-type (WT) on-target genomic sites. Intended single base edits in cyan, additional base substitutions in blue and protospacer adjacent motifs (PAMs) in brown. (b,c) On-target single base editing frequencies (b) and indel frequencies (c) induced by pegRNAs shown in (a) under PE3 setting. (d) Statistical analysis of normalized editing frequencies, setting the ones induced by regular pegRNAs as 1. n=18 at six on-target sites from three independent experiments shown in (b). (e) Sequences of PBS and RTT of pegRNAs and spegRNAs for generating pathogenic point mutations and WT on-target genomic sites. Pathogenic point mutations in red and same-sense mutations in blue. (f,g) On-target single base editing frequencies (f) and indel frequencies (g) induced by indicated pegRNAs and spegRNAs shown in (e) under PE3 setting. (h) Statistical analysis of normalized editing frequencies, setting the ones induced by regular pegRNAs as 1. n=9 at three on-target sites from three independent experiments shown in (f). (i) Sequences of PBS and RTT of pegRNAs and spegRNAs for repairing disease-associated mutations and on-target genomic sites containing pre-installed mutations. Correct bases in green, pre-installed point mutations in red and same-sense mutations in blue. (j,k) On-target single base editing frequencies (j) and indel frequencies (k) induced by indicated pegRNAs and spegRNAs shown in (i) under PE3 setting. (1) Statistical analysis of normalized editing frequencies, setting the ones induced by regular pegRNAs as 1. n=9 at three on-target sites from three independent experiments shown in (j). (b,c,f,g,j,k) Means±s.d. are from three independent experiments. (d,h,l) P value, Wilcoxon one-tailed signed-rank test. The median and interquartile range (IQR) are shown.

FIG. 15. pegRNA with stabilized secondary structure induced higher editing efficiency of indel. (a) Schematic diagrams illustrate predicted secondary structures of regular pegRNA, epegRNA-1 and epegRNA-2. Presumably, the free swing of RT-template and primer-binding site can break up the small hairpin (grey-shadowed, left panel), which destabilizes pegRNA. However, engineering within the small hairpin of pegRNAs could stabilize the secondary structures of epegRNA-1 and epegRNA-2 (middle and right panels, respectively). (b) On-target indel editing frequencies induced by pegRNA, epegRNA-1 and epegRNA-2 at indicated sites under PE3 setting or from non-transfected (NT) cells. Means s.d. are from three independent experiments. (c) Statistical analysis of normalized editing frequencies, setting the ones induced by regular pegRNAs as 1. n=93 indel edits at six on-target sites from three independent experiments shown in (b). P value, Wilcoxon one-tailed signed-rank test. The median and interquartile range (IQR) are shown.

FIG. 16. Structure-guided ACE2 editing. a, Interface inspection identifies potential sites for editing on hACE2. Representative structure (PDBid:6m17) of hACE2 (light cyan) in complex with SARS-CoV-2 RBD (grey) are shown as ribbons, with structural motifs at the interface highlighted in cyan on hACE2 and salmon on RBD (labeled as RBM). The interface is further detailed in three close-up views corresponding to three different clusters of the interface residues (cluster 1-3 depicted with orange, red and purple dashed boxes respectively). Key contacting residues were shown as stick models, polar and electrostatic interactions are indicated with black dashed lines. b, Foot-printing of RBD (grey) on the surface presentation of hACE2 (cyan). hACE2 residues involved in binding were labeled and boxed as in (a) to illustrate the three clusters. N-glycans on hACE2 are depicted as red and yellow spheres. c-e, Efficiency of PE3-mediated editing of individual hACE2 residues from cluster 1 (c), 2 (d) and 3 (e). f, Efficiency of PE-mediated simultaneous editing of hACE2 residues from cluster 1 and 2 with one pegRNA. c-f, Data are shown as means±s.d. (n=3).

FIG. 17. Specific mutations rendered hACE2 resistant to the binding of RBDs from globally prevalent SARS-CoV-2 strains. a, Interactions between selected hACE2 mutants (>30% editing efficiency) and immobilized SARS-CoV-2 RBD (wild-type strain) were determined with SPR, wherein AKA (Q24A/D30K/K31A), AAA (Q24A/D30K/K31A), K353del, AKA/K353del and AAA/K353del completely eliminated the interaction between the two. b, The angiotensin II hydrolyzing activity were not affected at all by indicated hACE2 mutants. Data are shown as means±s.d. (n=3). P value are from two-tail unpaired t test. c, Interactions between selected hACE2 mutants and immobilized SARS-CoV-2 RBD (B.1.1.7, B.1.351 and P.1 strains) were determined with SPR. Mutants K353del, AKA/K353del and AAA/K353del are resistant to the binding of RBDs from all strains tested.

FIG. 18. Cell surface interaction between hACE2 and RBDs from globally prevalent SARS-CoV-2 strains were blocked by selected hACE2 mutations. a-d, Representative flow cytometry plots showing the cell surface interaction of wildtype (WT) hACE2 or hACE2 mutants with the RBDs from globally prevalent SARS-CoV-2 strains, including WT (a), B.1.1.7 (b), B.1.351(c) or P.1(d). hACE2-KO 293FT cells (No hACE2) were used as a negative control while hACE2-KO 293FT cells overexpressing WT hACE2 (WT hACE2) served as positive controls. e, Proportion of 293FT cells exhibiting a detectable response to RBDs from indicated SARS-CoV-2 strains. Data are shown as means±s.d. (n=3). P value are from two-tail unpaired t test.

FIG. 19. Editing hACE2 prevented the entry of different pseudotyped SARS-CoV-2 strains. a, hACE2-KO 293FT cells exogenously overexpressing WT hACE2 or its indicated mutants were infected with different pseudoviruses, the entry efficiency of corresponding pseudovirus into hACE2-KO 293FT cells exogenously overexpressing WT hACE2 was taken as 100%. b, The efficiency of PE3-mediated editing of EF1aP-KI 293FT cells at indicated target sites. c, EF1aP-KI 293FT cells were edited by PE3 at indicated sites and then infected with different pseudoviruses. The entry efficiency of corresponding pseudovirus into mock-edited EF1aP-KI 293FT cells was taken as 100%. a & c, The four types of pseudoviruses each correspond to the WT, B.1.1.7, B.1.351 and P.1 SARS-CoV-2 strains. Pseudovirus entry efficiency was characterized as luciferase activity accompanying entry. Data are shown as means±s.d. (n=3). P value are from two-tail unpaired t test.

FIG. 20. Broad-spectrum anti-viral effects of hACE2 mutated at selected sites. a, Mutants K353del, AKA/K353del and AAA/K353del even rendered hACE2 resistant to the binding of RBDs from HCoV-NL63 and SARS-CoV, suggesting the broad-spectrum anti-viral effects of these mutations. b-c, Representative flow cytometry plots showing the cell surface interaction of WT hACE2 or hACE2 mutants with the RBDs from SARS-CoV (b) or HCoV-NL63 (c). hACE 2-KO 293 FT cells (No hACE2) were used as a negative control while hACE 2-KO 293 FT cells overexpressing WT hACE2 (WT hACE2) served as positive controls. d, Proportion of 293FT cells exhibiting a detectable response to RBDs from SARS-CoV or HCoV-NL63. Data are shown as means±s.d. (n=3). P value are from two-tail unpaired t test. e, Model of hACE2-editing mediated-prevention of hCoV infection.

FIG. 21 shows that pegRNA containing additional base substitutions induced higher efficiencies of intended single-base substitution. (A) Statistical analysis of normalized single-base editing frequencies induced by pegRNAs containing the indicated number of additional base substitutions, setting the frequencies induced by regular pegRNAs (without additional base substitution) to 1. (B) Heatmaps show the normalized single-base editing efficiency induced by the pegRNAs with one additional base substitution at the indicated positions. (C) Statistical analysis of normalized single-base editing frequencies induced by pegRNAs containing one additional base substitution at the indicated position, setting the frequencies induced by regular pegRNAs to 1. (D) Heatmaps show the normalized single-base editing efficiency induced by the pegRNAs with two additional base substitutions at the indicated position. (E) Statistical analysis of normalized single-base editing frequencies induced by pegRNAs containing two additional base substitutions at the indicated positions, setting the frequencies induced by regular pegRNAs to 1.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

### Enhanced Prime Editing

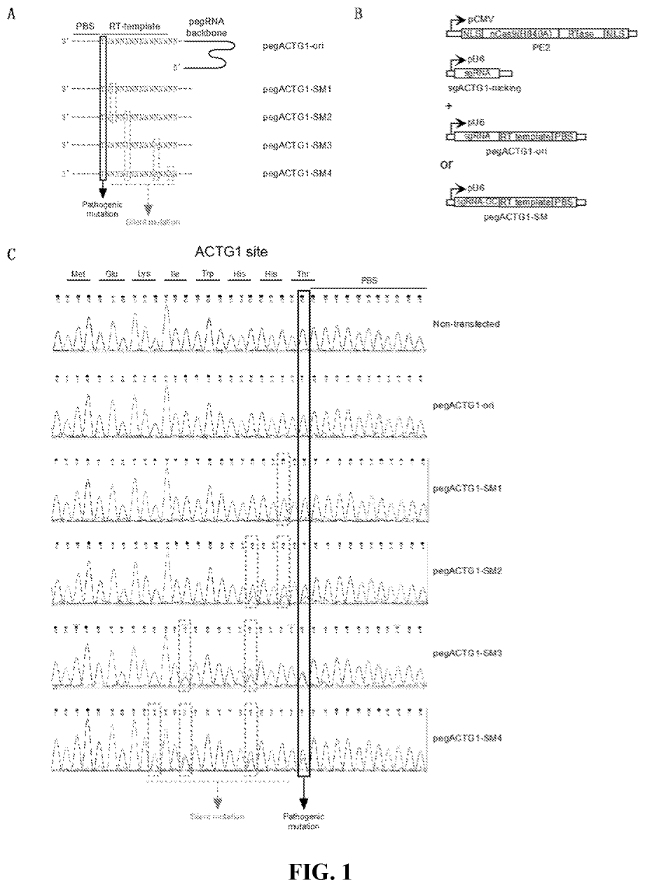

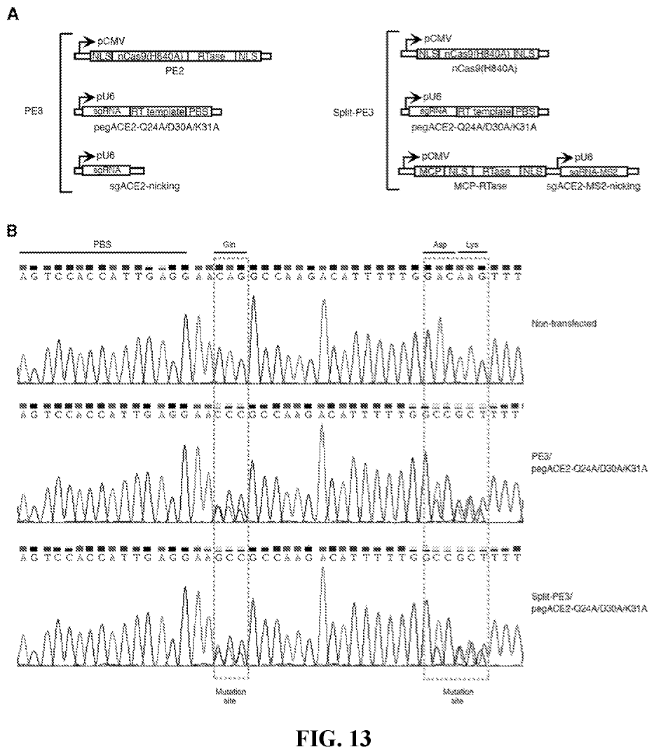

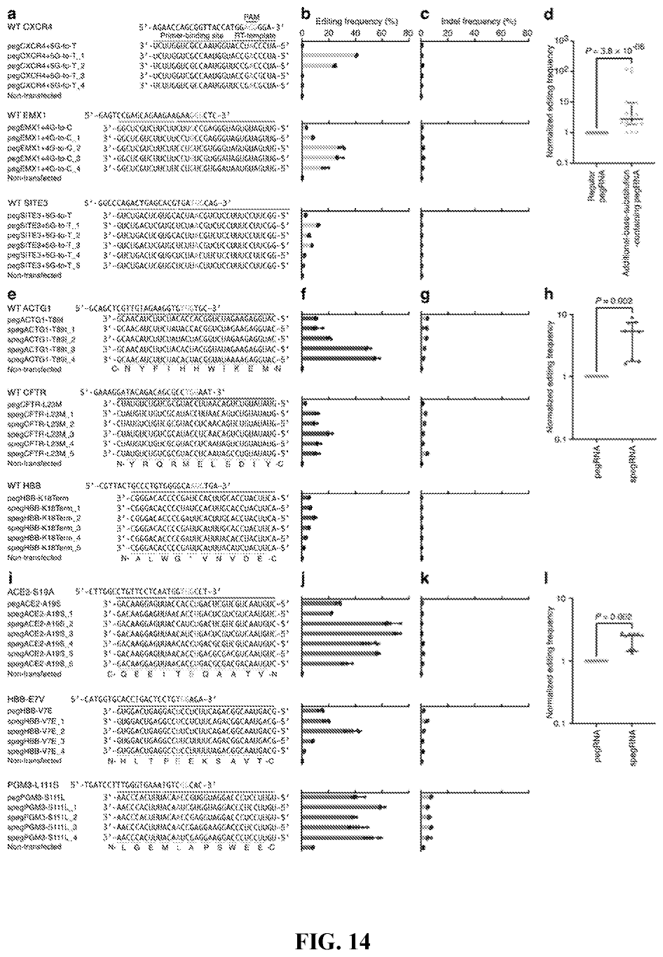

As shown in experimental Example 1 and FIG. 1-4, and Example 2 and FIG. 13-14, when one or more mutations were introduced to the prime editing gRNA (pegRNA), the editing efficiency of prime editing on a target mutation was greatly enhanced. The results also suggest that up to four mutations, in particular two mutations, have greater impact than just one silent mutation (Example 6). Also, when the mutations are spread out more (e.g., dual mutations at positions 1/4, 2/5 or 3/6), they appear to have stronger effect than when the silent mutations are adjacent the target mutation. If these additional mutations are designed to not impact the biological activity of the target protein, such as silent mutations and conservative mutations, then the incorporation of these mutations can have the net effect of increasing the efficiency of editing the target amino acid(s).

In one embodiment, therefore, provided is a method for generating a target mutation to a protein in a cell. In some embodiments, the method comprises introducing to the cell a prime editing system, wherein the prime editing system comprises a fusion protein comprising a nickase and a reverse transcriptase, and a prime editing guide RNA (pegRNA) encoding the target mutation and one or more silent or conservative mutations. Stated differently, the pegRNA can encode two or more mutations, at least one or two of these mutations are silent (or conservative) mutations. The remaining mutation(s) are the target mutation(s).

Prime editing is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

The pegRNA is capable of identifying the target nucleotide sequence to be edited, and encodes new genetic information that replaces the targeted sequence. The pegRNA consists of an extended single guide RNA (sgRNA) containing a primer binding site (PBS) and a reverse transcriptase (RT) template sequence. During genome editing, the primer binding site allows the 3′ end of the nicked DNA strand to hybridize to the pegRNA, while the RT template serves as a template for the synthesis of edited genetic information.

The fusion protein, in some embodiments, includes a nickase fused to a reverse transcriptase. An example nickase is Cas9 H840A. The Cas9 enzyme contains two nuclease domains that can cleave DNA sequences, a RuvC domain that cleaves the non-target strand and a HNH domain that cleaves the target strand. The introduction of a H840A substitution in Cas9, through which the histidine residue at 840 is replaced by an alanine, inactivates the HNH domain. With only the RuvC functioning domain, the catalytically impaired Cas9 introduces a single strand nick, hence a nickase.

Non-limiting examples of reverse-transcriptases include human immunodeficiency virus (HIV) reverse-transcriptase, moloney murine leukemia virus (M-MLV) reverse-transcriptase and avian myeloblastosis virus (AMV) reverse-transcriptase.

In some embodiments, the prime editing system further includes a single guide RNA (sgRNA) that directs the Cas9 H840A nickase portion of the fusion protein to nick the non-edited DNA strand.

Prime editing can be carried out by transfecting cells with the pegRNA and the fusion protein. Transfection is often accomplished by introducing vectors into a cell. Once internalized, the fusion protein nicks the target DNA sequence, exposing a 3′-hydroxyl group that can be used to initiate (prime) the reverse transcription of the RT template portion of the pegRNA. This results in a branched intermediate that contains two DNA flaps: a 3′ flap that contains the newly synthesized (edited) sequence, and a 5′ flap that contains the dispensable, unedited DNA sequence. The 5′ flap is then cleaved by structure-specific endonucleases or 5′ exonucleases. This process allows 3′ flap ligation, and creates a heteroduplex DNA composed of one edited strand and one unedited strand. The reannealed double stranded DNA contains nucleotide mismatches at the location where editing took place.

Silent mutations are mutations in DNA or RNA that do not have an observable effect on the organism's phenotype. They are a specific type of neutral mutation. In some embodiments, a silent mutation is a synonymous mutation. In some embodiments, a silent mutation affects only noncoding DNA (e.g., a mutation in an intron and does not affect RNA splicing). In some embodiment, a silent mutation produces a different amino acid but the altered amino acid has similar functionality to the original amino acid (e.g., a mutation producing leucine instead of isoleucine, or arginine instead of lysine). Such a silent mutation is sometime referred to as a conservative mutation.

Non-limiting examples of conservative mutations are provided in the table below, where a similarity score of 0 or higher indicates conservative mutation between the two amino acids.

TABLE A
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

In some embodiments, pegRNA encodes at least 1, or 2, or 3, or 4, or 5, or 6, or 7, or 8, or 9, or 10 silent mutations. In some embodiments, pegRNA encodes no more than 3, or 4, or 5 or 6 silent mutations. In some embodiments, the target mutation on the protein is encoded by a single nucleotide (or base) change (the target mutation on the polynucleotide). In some embodiments, at least one of the silent mutations is within 20 nucleotides from the target mutation on the polynucleotide. In some embodiments, at least one of the silent mutations is within 10, 15, 20, 25, 30, 35, 40, 45 or 50 nucleotides from the target mutation on the polynucleotide. In some embodiments, at least two of the silent mutations are within 10, 15, 20, 25, 30, 35, 40, 45 or 50 nucleotides from the target mutation on the polynucleotide. In some embodiments, at least three of the silent mutations are within 10, 15, 20, 25, 30, 35, 40, 45 or 50 nucleotides from the target mutation on the polynucleotide. In some embodiments, all of the silent mutations are within 10, 15, 20, 25, 30, 35, 40, 45 or 50 nucleotides from the target mutation on the polynucleotide.

In some embodiments, at least one of the silent mutations is at least 2 nucleotides away from the target mutation. In some embodiments, at least one of the silent mutations is at least 3 or 4 nucleotides away from the target mutation. In some embodiments, at least two of the silent mutations each is in a different codon from other mutations. In some embodiments, at least three of the silent mutations each is in a different codon from other mutations.

In some embodiments, at least one of the silent mutations is on the opposite side of the target mutation from another silent mutation. In some embodiments, all of the silent mutations are on the same side of the target mutation, 5′ or 3′.

The fusion protein and the pegRNA can each, independently, be introduced to the cell. The fusion protein may be introduced as a protein, or by a vector that encodes the fusion protein. The pegRNA may be introduced as an RNA, or by a vector that encodes the pegRNA. The two vectors may be separately introduced, to together in the same transfection, or combined as a single vector, without limitation.

In some embodiments, the fusion protein can further include other fragments, such as and nuclear localization sequences (NLS).

A “nuclear localization signal or sequence” (NLS) is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A non-limiting example of NLS is the internal SV40 nuclear localization sequence (iNLS).

### Split Prime Editing

Various “split” prime editing systems are also described here, which allow the nickase and the reverse transcriptase to be packaged into separate delivery vehicles (e.g., adeno-associated virus, AAV).

A typical AAV vehicle has a 4.7 kb capacity, but a coding sequence for the nickase (e.g., nCas-H840A) protein with a promoter and a poly-A signal is already about 4.7 kb in length. A fusion between the nickase and a reverse transcriptase requires a coding sequence that is about 7.3 kb, which cannot be packaged in an AAV. The other components, e.g., pegRNA, cannot be packaged together with the nickase in the same AAV either.

A conventional prime editing system, e.g., PE2, includes a nickase-RTase fusion and a pegRNA. In an example split system, a first construct encodes the nickase protein, a second construct encodes the RTase and the pegRNA. Further, in the second construct, the pegRNA includes one or two “tag sequence” and the RTase is fused to an RNA recognition peptide that is able to bind to the tag sequence. These two constructs can then be packaged in two separate AAV.

Upon introduction into a cell, the first construct from the first AAV expresses the nickase, and the second construct from the second AAV expresses the RTase-RNA recognition peptide and the pegRNA that includes the tag sequence. The RTase is recruited by the pegRNA, through the tag-RNA recognition peptide binding, and thus comes in contact with the nickase.

A PE3 system further includes sgRNA molecule (see, e.g., FIG. 11A). It has been shown that the edit inserted by PE2 can still be removed due to DNA mismatch repair of the edited strand. To avoid this problem during DNA heteroduplex resolution, an additional single guide RNA (sgRNA) is introduced in PE3. This sgRNA is designed to complement to the sequence of edited DNA strand by the pegRNA, but not the unedited DNA strand. It directs the nickase portion of the fusion protein to nick the unedited strand at a nearby site, opposite to the original nick. Nicking the non-edited strand causes the cell's endogenous repair system to copy the information in the edited strand to the complementary strand, permanently installing the edit.

In an example split PE3 system of the present disclosure (see illustration in FIG. 11B), a first construct encodes the nickase protein, a second construct (or a second set of constructs to be packaged in a single AAV) encodes the RTase, the pegRNA and the sgRNA. Further, in the second construct, the sgRNA includes one or two “tag sequence” (e.g., MS2) and the RTase is fused to an RNA recognition peptide (e.g., MCP) that is able to bind to the tag sequence. The set of constructs can then be packaged in two separate AAV.

Upon introduction into a cell, the first construct from the first AAV expresses the nickase, and the second construct from the second AAV expresses the RTase-RNA recognition peptide, the pegRNA, and the sgRNA that includes the tag sequence. The RTase is recruited by the sgRNA, through the tag-RNA recognition peptide binding, and thus comes in contact with the nickase.

In another embodiment, the tag sequence is embedded in the pegRNA, rather than in the sgRNA. Compared to the split PE3 configuration in which the tag is included in the pegRNA, this configuration is better suited in situations where the edited base is more distant from the pegRNA binding site.

Pairs of tag sequences and corresponding RNA recognition peptides are well known in the art. Examples include MS2/MS2 coat protein (MCP), PP7/PP7 coat protein (PCP), and boxB/boxB coat protein (N22p), the sequences of which are provided in Table B.

TABLE B
Example Tag-RNA Recognition Peptide Sequences

| | | SEQ ID |
|---|---|---|
| Name | Sequence | NO: |
| MS2 | ACAUGAGGAUCACCCAUGU | 3 |
| MS2 coat protein | MASNFTQFVLVDNGGTGDVTVAPSNFANGIAEWISSNSRSQAYKVTCSV | 4 |
| (MCP) | RQSSAQNRKYTIKVEVPKGAWRSYLNMELTIPIFATNSDCELIVKAMQG | |
| | LLKDGNPIPSAIAANSGIY | |
| PP7 | GGAGCAGACGAUAUGGCGUCGCUCC | 5 |
| PP7 coat protein | MGSKTIVLSVGEATRTLTEIQSTADRQIFEEKVGPLVGRLRLTASLRQN | 6 |
| (PCP) | GAKTAYRVNLKLDQADVVDSGLPKVRYTQVWSHDVTIVANSTEASRKSL | |
| | YDLTKSLVATSQVEDLVVNLVPLGR | |
| boxB | GCCCUGAAGAAGGGC | 7 |
| boxB coat protein | MGNARTRRRERRAEKQAQWKAAN | 8 |
| (N22p) | | |

With the split prime editing systems, methods for conducting genetic editing in a cell at a target site are also provided. In some embodiments, the method entails introducing to the cell a first viral particle enclosing a first construct encoding a nickase, and a second viral particle enclosing a second construct encoding a reverse-transcriptase fused to an RNA recognition peptide. In some embodiments, the second construct further encodes a pegRNA comprising an RNA recognition site (tag sequence) that the RNA recognition peptide binds to.

### Method of Genetic Editing and Prevention or Treatment of Coronavirus Infections

The present disclosure also provides compositions and methods for altering the sequence and activity of the human ACE2 (angiotensin-converting enzyme 2), such that it retains the enzymatic activity required by the body but is unable to be bound by the spike (S) protein of the coronaviruses. As such, cells with the mutated ACE2 would be resistant to infection by the coronavirus.

As shown in Examples 3-4, mutations at S19, Q24, D30, K31, and K353 of the human ACE2 protein (positions according to SEQ ID NO:1) can reduce or abolish its binding to the spike protein. Example mutations tested include, without limitation, S19A, Q24A, D30A, D30K, K31A, and K353del.

In one embodiment, therefore, the present disclosure provides compositions and methods useful for introducing one or more such mutations to the ACE2 protein. In some embodiments, the composition includes a prime editing system that includes a suitably design pegRNA for generating the mutation. In some embodiments, the pegRNA includes a fragment that (a) is capable of hybridizing to (a portion of) the genomic sequence of human ACE2 and (b) encodes a target mutation at one or more residues selected from the group consisting of S19, Q24, D30, K31, or K353, which positions are according to SEQ ID NO:1.

Example mutations (on the pegRNA) include C70A, C70G, A71C, G72C, G88A, A89C, C90G, A91G, A92C, G93C, A113C, A₁₀₅₇A₁₀₅₈G₁₀₅₉->del and the combinations thereof (positions according to SEQ ID NO:2).

In some embodiments, the target mutation is selected from Q24, D30, K31, or K353 or the combinations thereof. In some embodiments, the target mutation is at Q24. In some embodiments, the target mutation is at D30. In some embodiments, the target mutation is at K31. In some embodiments, the target mutation is at K353. In some embodiments, the target mutation is at Q24, D30 and K31. In some embodiments, the target mutation is at Q24, D30, K31 and K353. In some embodiments, the mutations are non-conservative mutations. For instance, D is not mutated to E and K is not mutated to R. In some embodiments, K353 is mutated to any amino acid other than R. In some embodiments, K353 is deleted. Example target mutations are Q24A/D30A/K31A, Q24A/D30K/K31A, Q24A/D30A/K31A/K353del, and Q24A/D30K/K31A/K353del.

Mutant ACE2 proteins and fragments are also provided, in some embodiments. The mutant ACE2 protein or fragment, in some embodiments, includes one or more mutations at residues selected from the group consisting of S19, Q24, D30, K31, or K353, which positions are according to SEQ ID NO:1. In some embodiments, the mutations are non-conservative mutations. For instance, D is not mutated to E and K is not mutated to R. In some embodiments, K353 is mutated to any amino acid other than R. In some embodiments, K353 is deleted. Example mutations are Q24A/D30A/K31A, Q24A/D30K/K31A, Q24A/D30A/K31A/K353del, and Q24A/D30K/K31A/K353del.

Also provided, in some embodiments, are polynucleotides that encode such mutant ACE2 proteins or fragments. Still provided are cells that include such mutant ACE2 proteins or fragments, and methods of introducing such mutations into a wild-type ACE2 coding sequence into the genome of a target cell. Such methods may be gene editing or introduction of a polynucleotide that encodes the mutant protein or fragment thereof.

In some embodiments, the pegRNA further includes one or more silent mutations (not changing other, non-target, amino acids (or only changing to a similar one).

The prime editing system can further include a fusion protein comprising a nickase and a reverse transcriptase. The prime editing system may be introduced to a cell in vitro or in vivo, by itself, or through encoding vectors.

Also provided are compositions and methods for treating or preventing infection by a coronavirus. In some embodiments, the method entails administering to a subject one or more polynucleotides encoding the prime editing system as disclosed herein. In some embodiment, the coronavirus is SARS-CoV-2 (severe acute respiratory syndrome coronavirus 2) or SARS-CoV. In some embodiments, the subject has symptoms of Coronavirus Disease 2019 (COVID-19).

Methods of delivering vectors to a host cell, whether in vitro or in vivo, are known in the art. A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. In some embodiments of the present disclosure the vector is an episomal vector, i.e., a non-integrated extrachromosomal plasmid capable of autonomous replication. In some embodiments, the episomal vector includes an autonomous DNA replication sequence, i.e., a sequence that enables the vector to replicate, typically including an origin of replication (OriP). In some embodiments, the autonomous DNA replication sequence is a scaffold/matrix attachment region (S/MAR). In some embodiments, the autonomous DNA replication sequence is a viral OriP. The episomal vector may be removed or lost from a population of cells after a number of cellular generations, e.g., by asymmetric partitioning. In some embodiments, the episomal vector is a stable episomal vector and remains in the cell, i.e., is not lost from the cell. In some embodiments, the episomal vector is an artificial chromosome or a plasmid. In some embodiments, the episomal vector comprises an autonomous DNA replication sequence. Examples of episomal vectors used in genome engineering and gene therapy are derived from the Papovaviridae viral family, including simian virus 40 (SV40) and BK virus; the Herpesviridae viral family, including bovine papilloma virus 1 (BPV-1), Kaposi's sarcoma-associated herpesvirus (KSHV), and Epstein-Barr virus (EBV); and the S/MAR region of the human interferon b gene. In some embodiments, the episomal vector is an artificial chromosome. In some embodiments, the episomal vector is a mini chromosome.

The term “vector” includes both viral and non-viral means for introducing the nucleic acid into a cell in vitro, ex vivo, or in vivo. A large number of vectors known in the art may be used to manipulate nucleic acids, incorporate response elements and promoters into genes, etc. Possible vectors include, for example, plasmids or modified viruses including, for example, bacteriophages such as lambda derivatives, or plasmids such as PBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited, to retrovirus, adenovirus adeno-associated virus, pox, baculovirus, vaccinia, herpes simplex, Epstein-Barr, adenovirus, geminivirus, and caulimovirus vectors. Retroviral vectors have emerged as a tool for gene therapy by facilitating genomic insertion of a desired sequence. Retroviral genomes (e.g., murine leukemia virus (MLV), feline leukemia virus (FLV), or any virus belonging to the Retroviridae viral family) include long terminal repeat (LTR) sequences flanking viral genes. Upon viral infection of a host, the LTRs are recognized by integrase, which integrates viral genome into the host genome. A retroviral vector for targeted gene insertion does not have any of the viral genes, and instead has the desired sequence to be inserted between the LTRs. The LTRs are recognized by integrase and integrates the desired sequence into the genome of the host cell.

In some embodiments, the viral vector is an AAV (adeno-associated virus) vector. The genomic organization of all known AAV serotypes is similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP-1, -2 and -3) form the capsid. The terminal 145 nt are self-complementary and are organized so that an energetically stable intramolecular duplex forming a T-shaped hairpin may be formed.

An AAV vector herein refers to a vector comprising one or more polynucleotide sequences of interest, a gene product of interest, genes of interest or “transgenes” that are flanked by at least one parvoviral or AAV inverted terminal repeat sequences (ITRs). Such rAAV vectors can be replicated and packaged into infectious viral particles when present in an insect host cell that is expressing AAV rep and cap gene products (i.e., AAV Rep and Cap proteins). Typically, a gene product of interest is flanked by AAV ITRs on either side. Any AAV ITR may be used in the constructs of the invention, including ITRs from AAV1, AAV2, AAV4, AAV5, AAV6, AAV7, AAV8, AAV9, AAV10, AAV11 and/or AAV12.

An AAV vector for use in the present technology may be produced either in mammalian cells or in insect cells. Both methods are described in the art. For example, Grimm et al. (2003 Molecular Therapy 7(6):839-850) disclose a strategy to produce AAV vectors in a helper virus free and optically controllable manner, which is based on transfection of only two plasmids into 293T cells.

Each serotype of AAV may be more suitable for one or more particular tissues. For instance, AAV2, AAV3, AAV4, AAV5, AAV7 and AAV8 may be suitable for retina; AAV1, AAV2, AAV4, AAV5, AAV7 and AAV10 may be suitable for neurons; AAV2, AAV4, AAV8 and AAV9 may be suitable for the brain; AAV3, AAV5, AAV6, AAV9 and AAV10 may be suitable for the lung; AAV1, AAV6, AAV9 and AAV10 may be suitable for the heart; AAV2, AAV3 and AAV6-10 may be suitable for the liver; all of the serotypes except AAV5 may be suitable for muscle tissues; AAV2 and AAV10 may be suitable for the kidney; and AAV1, AAV7 and AAV9 may be suitable for the pancreas.

Non-viral vectors include, but are not limited to, plasmids, liposomes, electrically charged lipids (cytofectins), DNA-protein complexes, and biopolymers. In addition to a nucleic acid, a vector may also include one or more regulatory regions, and/or selectable markers useful in selecting, measuring, and monitoring nucleic acid transfer results (transfer to which tissues, duration of expression, etc.).

Transposons and transposable elements may be included on a vector. Transposons are mobile genetic elements that include flanking repeat sequences recognized by a transposase, which then excise the transposon from its locus at the genome and insert it at another genomic locus (commonly referred to as a “cut-and-paste” mechanism). Transposons have been adapted for genome engineering by flanking a desired sequence to be inserted with the repeat sequences recognizable by transposase. The repeat sequences may be collectively referred to as “transposon sequence.” In some embodiments, the transposon sequence and a desired sequence to be inserted are included on a vector, the transposon sequence is recognized by transposase, and the desired sequence can then be integrated into the genome by the transposase.

Vectors may be introduced into the desired host cells by known methods, including, but not limited to, transfection, transduction, cell fusion, and lipofection. Vectors can include various regulatory elements including promoters. In some embodiments, the present disclosure provides an expression vector including any of the polynucleotides described herein, e.g., an expression vector including polynucleotides encoding the fusion protein and/or the pegRNA.

### EXAMPLES

### Example 1. Silent Mutations Increased Editing Efficiency

This example tested whether pegRNAs containing additional, silent mutations could increase editing efficiency.

This example compared the prime editing efficiencies induced by the canonical pegRNA that only contained an intended edit and pegRNAs that contained the intended edit plus one or a few silent mutations (FIGS. 1A, 2A, 3A and 4A) when using the primer editors (PE) to generate pathogenic point mutations (FIGS. 1B, 2B, 3B and 4B). It was found that the canonical pegRNA induced only low levels of intended pathogenic point mutations, but the pegRNAs that contained silent mutations induced higher levels of pathogenic mutations (FIGS. 1C, 2C, 3C and 4C).

The design of the first experiment is illustrated in the schematics of FIG. 1A. The original pegRNA included a T89I mutation in the ACTG1 gene (pegACTG1-Ori). pegACTG1-SM1, pegACTG1-SM2, pegACTG1-SM3, and pegACTG1-SM4 each included one or more silent mutations in the downstream codons. pegACTG1-SM1 had one silent mutation; pegACTG1-SM2 had two silent mutations; pegACTG1-SM3 also had two silent mutations which were farther away from T89I than in pegACTG1-SM2; pegACTG1-SM4 had three silent mutations. The plasmids expressing PE2, sgACTG1-nicking and pegRNAs for inducing T89I mutation in ACTG1 gene are illustrated in FIG. 1B.

While the original pegRNA (pegACTG1-Ori) led to barely observable editing, the ones with silent mutations produced greatly higher editing efficiency (FIG. 1C). In particular, the editing efficiency of pegACTG1-SM1 at the target site (T89I) was a few folds higher than pegACTG1-Ori, and the editing efficiency of pegACTG1-SM2 was even higher. Also interestingly, pegACTG1-SM3 and pegACTG1-SM4 both resulted in a few fold higher editing efficiency at the target site (T89I) than pegACTG1-SM2, even though pegACTG1-SM3 and pegACTG1-SM2 both included two silent mutations. This suggests that both the number of added silent mutations and their spread can influence editing efficiency.

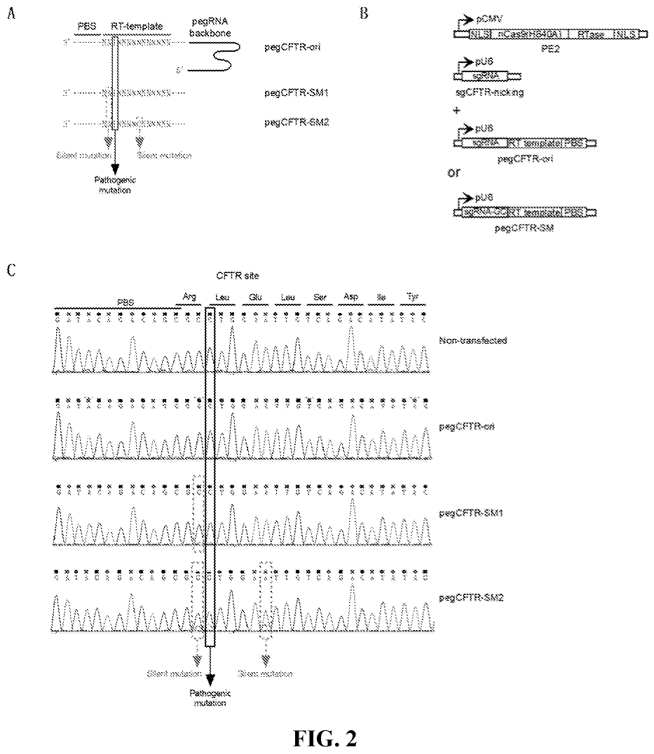

In a second experiment, illustrated in the schematics of FIG. 2A, the original pegRNA included a L23M mutation in the CFTR gene (pegCFTR-Ori). The silent mutations included a nearby nucleotide (pegCFTR-SM1) in a codon to the 3′ direction, or along with another nucleotide 5 nt away to the 5′ direction in another codon. The plasmids expressing PE2, sgACTG1-nicking and pegRNAs for inducing T89I mutation in CFTR gene are illustrated in FIG. 2B.

Like in the first experiment, while the original pegRNA (pegCFTR-Ori) led to barely observable editing, the ones with silent mutations produced greatly higher editing efficiency (FIG. 2C). In particular, the editing efficiency of pegCFTR-SM2 was a few folds higher than pegCFTR-SM1, which in turn is a few folds higher than pegCFTR-Ori.

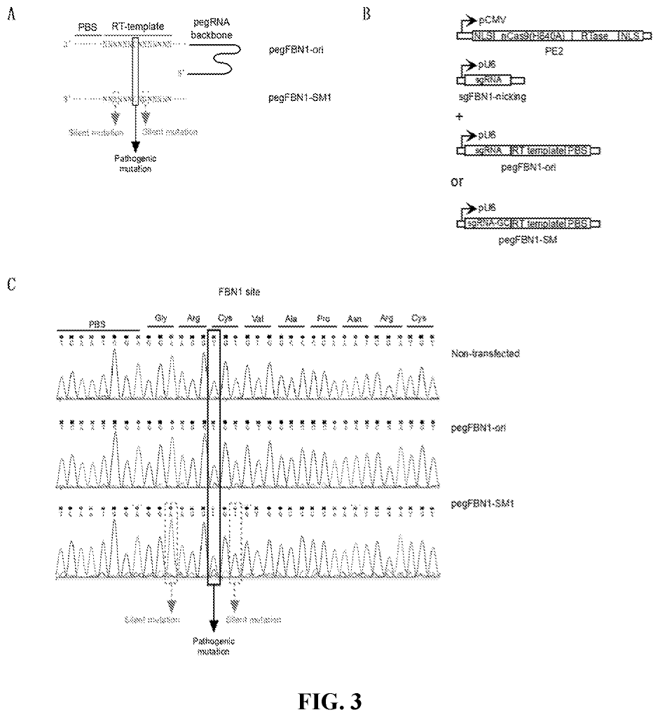

FIG. 3A-C present the design and results of editing a mutation in yet another gene, the C160G mutation in the FBN1 gene. Again, the nearby silent mutations (sgFBN1-SM1) greatly enhanced the editing efficiency (sgFBN1-Ori).

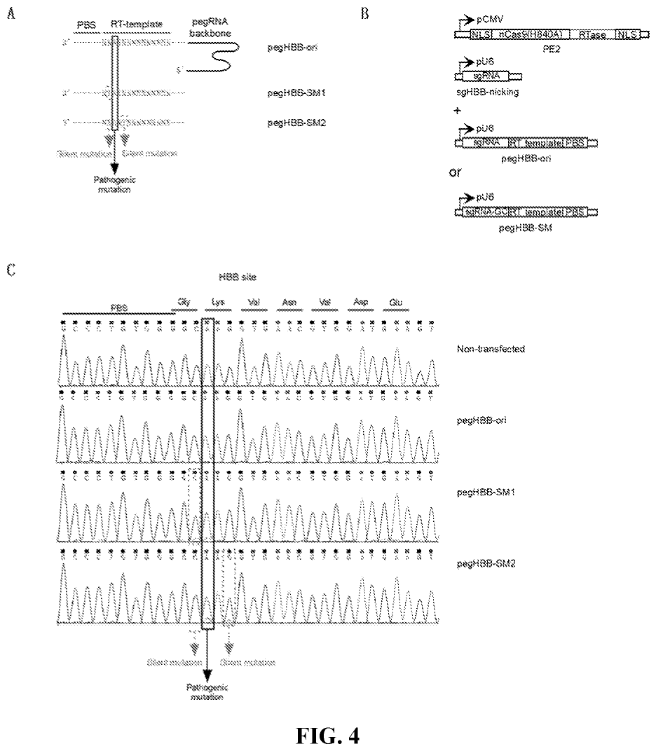

In yet another experiment, targeting the K18Ter mutation in the HBB gene, two pegRNA with silent mutations were individually tested for their effect in enhancing the editing efficiency of the target mutation. The silent mutation that is 2 nt away at the 5′ (regHBB-SM2) slightly outperformed the one that is adjacent to the target (1 nt away at the 3′ direction). See, FIG. 4A-C.

This example, therefore, demonstrates that the addition of silence mutations close the target editing site can greatly enhance the editing efficiency of prime editing. As silent mutations do not change the coding of amino acids, the edited genes that contain both pathogenic mutations and silent mutations can recapitulate the phenotypes caused by the pathogenic mutations. Similarly, the same strategy can also be used to correct pathogenic mutations, including the ones for therapeutic purpose.

### Example 2. Highly-Efficient Prime Editing by Applying Same-Sense Mutation in pegRNA or Stabilizing its Structure

This example expanded and confirmed the experiments of Example 1. This example shows that, by introducing additional base substitutions in prime editing guide RNA (pegRNA), the prime editor's single base substitution efficiency was increased upto 131.3-fold. Also, when the pegRNA secondary structure was stabilized with structural changes, the prime editor's indel efficiency was increased for upto 10.6-fold. These strategies enable efficient prime editing at previously-invalid target sites, including those associated with genetic disorders.

### Methods and Materials

### Plasmid Construction

The primer set (pegRNA_F/pegSITE3_R) was used to amplify the pegRNA-scaffold-fragment with template pGL3-U6-sgRNA-PGK-puromycin (addgene, 51133). Then the amplified pegRNA-scaffold-fragment was cloned into the BsaI and EcoRI linearized pGL3-U6-sgRNA-PGK-puromycin with NovoRec® plus One step PCR Cloning Kit (NR005, Novoprotein) to generate the vector pGL3-U6-pegRNA-PGK-puromycin for the expression of pegRNA.

Oligonucleotides CXCR4_FOR/CXCR4_REV were annealed and ligated into BsaI linearized pGL3-U6-pegRNA-PGK-puromycin to generate the vector psgCXCR4-spacer. Oligonucleotides CXCR4_5_FOR/CXCR4_5_REV were annealed and ligated into the PflFI and EcoRI linearized psgCXCR4-spacer to generate the vector ppegCXCR4+5G-to-T for the expression of pegCXCR4+5G-to-T. Other expression vectors for pegRNA and spegRNA were constructed by the similar strategy.

Oligonucleotides CXCR4_nick_FOR/CXCR4_nick_REV were annealed and ligated into BsaI linearized pGL3-U6-sgRNA-PGK-puromycin to generate the vector pnick-sgCXCR4 for the expression of nick-sgCXCR4. Other expression vectors for nick-sgRNA were constructed by the similar strategy.

The primer set (pegRNA_2024plusGC_F/pegRNA_2024plusGC_R) was used to insert a G/C pair in pGL3-U6-pegRNA-PGK-puromycin and generate pGL3-U6-epegRNA1-PGK-puromycin. The primer set (pegRNA_1629CG_F/pegRNA_1629CG_R) was used to change a G/A mismatch to a C/G pair in pGL3-U6-pegRNA-PGK-puromycin and generate pGL3-U6-epegRNA2-PGK-puromycin.

Oligonucleotides GCH1_FOR/GCH1_REV were annealed and ligated into BsaI linearized pGL3-U6-epegRNA1-PGK-puromycin and pGL3-U6-epegRNA2-PGK-puromycin to generate the vector psgGCH1-spacer-1 and psgGCH1-spacer-2. Oligonucleotides pegGCHl_+1GATins_FOR/pegGCHl_+1GATins_REV were annealed and ligated into the PflFI and EcoRI linearized psgGCH1-spacer-1 and psgGCH1-spacer-2 to generate the vector pepegGCHl_+1GATins-1 and pepegGCHl_+1GATins-2 for the expression of epegGCH1_+1GATins-1 and epegGCH1_+1GATins-2. Other expression vectors for epegRNA were constructed by the similar strategy.

### Cell Culture and Transfection

293FT (Thermo Fisher Scientific, R70007), U2OS (ATCC® HTB-96) and HeLa (ATCC® CCL-2™) cells were maintained in DMEM (10566, Gibco/Thermo Fisher Scientific)+10% FBS (16000-044, Gibco/Thermo Fisher Scientific) and regularly tested to exclude mycoplasma contamination.

For prime editing with pegRNA (spegRNA or epegRNA), 293FT and U2OS cells were seeded in a 24-well plate at a density of 1×10⁵ per well and transfected with 250 μl serum-free Opti-MEM that contained 2.6 μl LIPOFECTAMINE LTX (Life, Invitrogen), 1.3 μl LIPOFECTAMINE plus (Life, Invitrogen), 0.9 μg PE2 expression vector, 0.3 μg pegRNA (spegRNA or epegRNA) expression vector with 0.1 μg nick-sgRNA expression vector. After 24 hrs, puromycin (ant-pr-1, InvivoGen) was added to the medium at the final concentration of 4 μg/ml. After another 48 hrs, the genomic DNA was extracted from the cells with QuickExtract™ DNA Extraction Solution (QE09050, Epicentre) for subsequent sequencing analysis.

### Cell Line Construction

To establish ACE2-S19A cell line, the 293FT cells were seeded into a 60-mm plate at a density of 4×10⁵ per well and cultured for 24 hrs. Cells were transfected with plasmids expressing PE2, pegACE2-S19A and nick-sgACE2, according to the manufacturer's instruction. After 48 hrs, 10 μg/ml puromycin was added into the media for two days. ACE2-S19A cell line expanded from a single colon and was validated by genomic DNA sanger sequencing. HBB-E7V cell line was constructed by the similar strategy.

### DNA Library Preparation and Sequencing

Target genomic sequences were PCR amplified by Phanta® Max Super-Fidelity DNA Polymerase (P505, Vazyme) with primer sets flanking examined pegRNA target sites. Indexed DNA libraries were prepared by using the NEBNext Ultra II FS DNA Library Prep Kit for Illumina. After quantitated with Qubit High-Sensitivity DNA kit (Invitrogen), PCR products with different tags were pooled together for deep sequencing by using the Illumina HiSeq X10 (2×150) or NextSeq 500 (2×150) at Shanghai Institute Nutrition and Health, Big Data Center Omics Core, Shanghai, China. Raw read qualities were evaluated by FastQC (v0.11.8, www.bioinformatics.babraham.ac.uk/projects/fastqc/). For paired-end sequencing, only R1 reads were used. Adaptor sequences and read sequences on both ends with Phred quality score lower than 30 were trimmed. Clean reads were then mapped with the BWA-MEM algorithm (v0.7.17-r1188) to target sequences. After piled up with Samtools (v1.9), editing frequencies were further calculated.

### Base Substitution Frequency Calculation

Base substitutions were selected at each bases of the examined pegRNA target sites that were mapped with at least 1,000 independent reads, and obvious base substitutions were only observed at the targeted base editing sites. Base substitution frequencies were calculated by dividing base substitution reads (without indels) by total reads using CFBI pipeline (github.com/YangLab/CFBI, v1.0.0). respectively.

### Intended Indel Frequency Calculation

Intended indel frequencies were calculated as: (count of reads with only intended indel at the target site)/(count of total reads covering the target site). The edited reads containing intended indels were also requested not to carry any other point mutations or indels within the region spanning from upstream 8 nucleotides to the target site to downstream 52 nucleotides to PAM site (80 bp).

### Unintended Indel Frequency Calculation

Unintended indel frequencies were estimated among reads aligned in the region spanning from upstream 8 nucleotides to the target site to downstream 52 nucleotides to PAM site (80 bp). Unintended indel frequencies for base substitution were calculated according to reported CFBI pipeline (github.com/YangLab/CFBI, v1.0.0) as: (count of reads containing at least one unintended inserted and/or deleted nucleotide)/(count of total reads aligned in the estimated region). Unintended indel frequencies for targeted insertion/deletion were calculated as: (count of reads containing unintended indels)/(count of total reads aligned in the estimated region).

Indel Frequency Calculation at pegRNA-Dependent OT Site

Indel frequencies for pegRNA-dependent OT site insertion/deletion were estimated among reads aligned in the region spanning from upstream 8 nucleotides to OT site to downstream 52 nucleotides to PAM site (80 bp), and calculated according to reported CFBI pipeline (github.com/YangLab/CFBI, v1.0.0) as: (count of reads containing at least one unintended inserted and/or deleted nucleotide)/(count of total reads aligned in the estimated region).

### Results

### Introduction of Silent Mutation Enhanced Editing Efficiency

The instant inventors proposed to that introducing additional base substitutions into the RT-template of pegRNA can enhance the intended base substitution efficiency by PE3. We first compared prime editing efficiencies induced by regular pegRNAs that contain only an intended single base substitution with those by pegRNAs that contain the intended single base substitution and additional base substitutions (FIG. 14a). As shown in the figure, the inclusion of additional base substitutions within pegRNAs could induce higher editing efficiencies than regular pegRNAs (FIG. 14b), without affecting on-target indel frequencies (FIG. 14c). Of note, some optimal pegRNAs that contain additional base substitutions (e.g., pegCXCR4+5G-to-T_1, pegEMXl+4G-to-C_2, pegSITE3+5G-to-T_1, pegPNRP+6G-to-T_2, pegRUNX1+6G-to-C_2 and pegVEGFA+5G-to-T_1 in FIG. 14a) mediated upto 131.3-fold (FIG. 14d, average 22.4-fold) higher editing efficiency than their corresponding regular pegRNAs without additional base substitution.

Next, we applied this new PE system with additional-base-substitution-containing pegRNAs to induce single base substitutions to generate three pathogenic mutations (FIG. 14e-1h) or correct three pre-installed mutations, which are associated with human diseases (FIG. 14i-141). To avoid potential amino-acid changes, we introduced same-sense mutations, instead of random mutations, into the RT-template of pegRNAs. Different to relatively low efficiencies when using regular pegRNAs (FIGS. 14f and 14j), the application of same-sense-mutation-containing pegRNAs (spegRNAs) significantly increased editing efficiencies in generating or correcting pathogenic mutations by PE3 (FIGS. 14f and 14j), without influencing their on-target indel frequencies (FIGS. 14g and 14k).

In general, spegRNAs triggered maximally 8.9-fold (average 4.6-fold) enhancement of editing efficiencies when generating pathogenic mutations (FIG. 14h) or maximally 2.8-fold (average 2.3-fold) when repairing disease-associated mutations (FIG. 14l). In addition, we compared spegRNAs and regular pegRNAs for generating single base substitution, pathogenic point mutation or repairing pre-installed mutation in other human cells (U2OS and HeLa) and found that optimized spegRNAs induced obviously higher editing efficiencies than regular pegRNAs in these two cells as well. Systematical analysis of all the editing results from pegRNAs with additional base substitutions suggested that using two additional base substitutions could boost the highest editing efficiency across all the tested sites.

To further evaluate the editing precision of spegRNA, we determined byproducts at on-target sites and pegRNA-dependent off-target (OT) editing at predicted OT sites that have sequence similarity to on-target sites. No obvious byproduct was triggered by spegRNAs comparing to regular pegRNAs and no OT editing was detected at potential OT sites, demonstrating the editing precision with the use of spegRNA.

Stabilization of sgRNA Enhanced Editing Efficiency

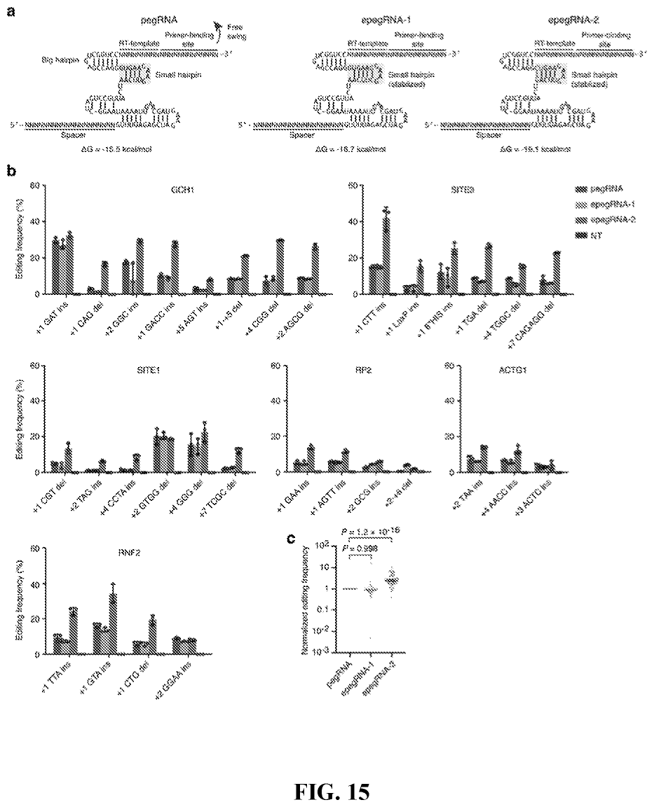

Another advantage of using PE is to introduce small indels at targeted sites. As small indels can be readily resolved by endogenous MMR, we sought to use an alternative strategy to enhance indel editing efficiency of PE3. Comparing to sgRNA, pegRNA contains two extra parts (primer-binding site and RT-template) at its 3′-end. We proposed that the small hairpin of regular pegRNA could be broken up by the free swing of primer-binding site and RT-template, thus compromising the secondary structure stability of pegRNA (FIG. 15a, left panel). Therefore, we set to engineer the backbone of pegRNA to stabilize its secondary structure by inserting a G/C pair (epegRNA-1) or changing a G/A mismatch to a C/G pair (epegRNA-2) in the small hairpin of pegRNA (FIG. 15a, middle and right panels, respectively).

We compared the indel editing efficiencies induced by regular pegRNAs with those by epegRNA-1 and epegRNA-2 for generating 31 types of small indels across six on-target sites under the PE3 setting in 293FT cells (FIG. 15b). Though epegRNA-1 showed similar indel editing efficiency as regular pegRNA, epegRNA-2 significantly improved the editing efficiency of PE3 system (FIG. 15c), with the maximal improvement upto 10.6-fold (average 3.0-fold).

We also examined unintended indel frequencies and byproducts at on-target sites and indel frequencies at predicted pegRNA-dependent OT sites and found that both epegRNA-1 and epegRNA-2 rarely trigger these unintended events comparing to regular pegRNAs, indicating the editing precision of epegRNAs. To further evaluate the efficacy of epegRNA in other cells, we compared the editing frequencies of regular pegRNAs and epegRNAs by inducing five types of indels across three sites in U2OS cells and found that epegRNA-2 significantly improved indel editing efficiencies as well.

As a versatile editing tool with high product purity and editing specificity, PE has great potentials in the application of correcting pathogenic mutations to treat genetic disorders. Though PE3 induced efficient editing at some target sites, its efficiency remained generally low at a lot of target sites, including the ones associated with human diseases (FIG. 14f, 14j). Here, we developed two strategies, spegRNAs by applying same-sense mutations or epegRNAs by stabilizing RNA secondary structure, to individually enhance editing efficiency of PE3 to generate single base substitutions or indels, across multiple target sites, including some pathogenic sites, in various human cells. Of note, both spegRNA and epegRNA strategies do not require extra protein or RNA component for the improvement and thus constrain the total size of PE3 system for in vivo delivery, such as viral delivery.

### Example 3. Editing of ACE2 to Prevent SARS-CoV-2 Infection

Since the end of 2019, COVID-19 has impacted the lives of millions of people worldwide. The disease-causing virus SARS-CoV-2 (severe acute respiratory syndrome coronavirus 2) enters cells by binding to the cell surface receptor ACE2 (angiotensin-converting enzyme 2) with its spike (S) protein. Thus, engineering ACE2 could be a potential way to prevent the infection of SARS-CoV-2.

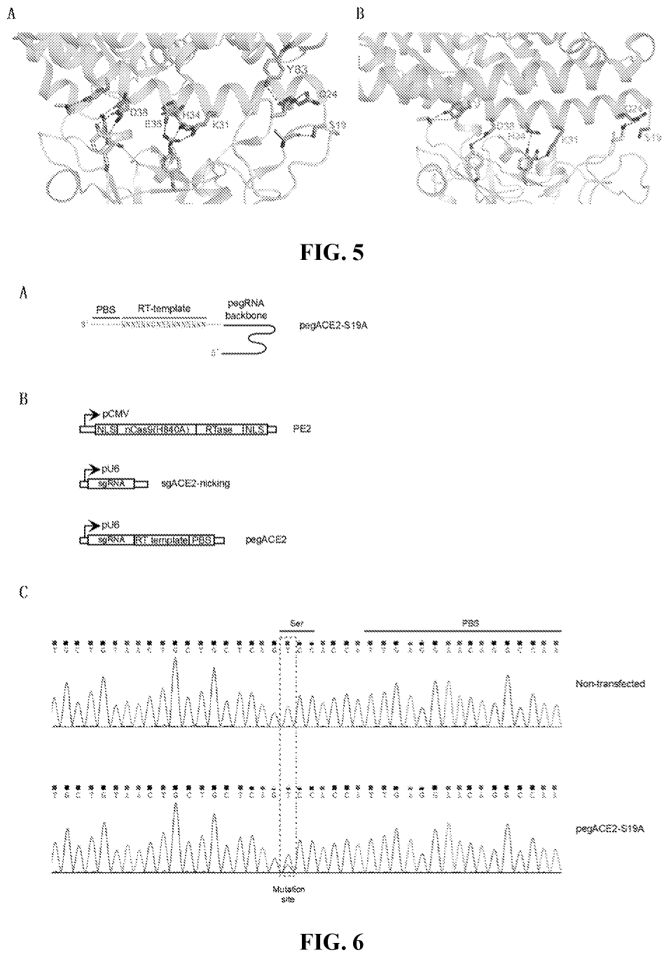

However, ACE2 is a transmembrane protein that can antagonize the effects of ACE on cardiovascular system and the deletion of ACE2 gene in a mouse model of lung injury can lead to more severe condition. Thus, the enzyme activity of ACE2 can protect lung tissue and the engineering of human ACE2 should maintain its activity. By analyzing the interface between human ACE2 and receptor binding domain (RBD) of SARS-CoV-2 spike (FIG. 5), this example selected the N-terminal region of ACE2 for engineering.

By co-transfecting primer editor 2 (PE2) with pairs of pegRNAs and nicking sgRNAs, this example tried to induce various mutations in the S-binding region of ACE2. It was found that the pair of pegACE2-S19A|sgACE2-nicking (FIG. 6A, 6B) can efficiently induce S19A (T55C base substitution in the coding region of ACE2 gene) mutation (FIG. 6C).

TABLE 1
Sequences of Human ACE2

Amino acid sequence (NP_068576.1) SEQ ID NO: 1 (signal peptide is underlined)

| | |
|---|---|
| 1 | MSSSSWLLLS LVAVTAAQST IEEQAKTFLD KFNHEAEDLF YQSSLASWNY NINITEENVQ |
| 61 | NMNNAGDKWS AFLKEQSTLA QMYPLQEIQN LTVKLQLQAL QQNGSSVLSE DKSKRINTIL |
| 121 | NTMSTIYSTG KVCNPDNPQE CLLLEPGLNE IMANSLDYNE RLWAWESWRS EVGKQLRPLY |
| 181 | EEYVVLKNEM ARANHYEDYG DYWRGDYEVN GVDGYDYSRG QLIEDVEHTE EEIKPLYEHL |
| 241 | HAYVRAKLMN AYPSYISPIG CLPAHLLGDM WGRFWINLYS LTVPFGQKPN IDVTDAMVDQ |
| 301 | AWDAQRIFKE AEKFFVSVGL PNMTQGEWEN SMLTDPGNVQ KAVCHPTAWD LGKGDFRILM |
| 361 | CTKVTMDDEL TAHHEMGHIQ YDMAYAAQPF LLRNGANEGF HEAVGEIMSL SAATPKHLKS |
| 421 | IGLLSPDFQE DNETEINELL KQALTIVGTL PFTYMLEKWR WMVFKGEIPK DQWMKKWWEM |
| 481 | KREIVGVVEP VPHDETYCDP ASLFHVSNDY SFIRYYTRTL YQFQFQEALC QAAKHEGPLH |
| 541 | KCDISNSTEA GQKLENMLRL GKSEPWTLAL ENVVGAKNMN VRPLLNYFEP LFTWLKDQNK |
| 601 | NSFVGWSTDW SPYADQSIKV RISLKSALGD KAYEWNDNEM YLFRSSVAYA MRQYFLKVKN |
| 661 | QMILFGEEDV RVANLKPRIS FNFFVTAPKN VSDIIPRTEV EKAIRMSRSR INDAFRLNDN |
| 721 | SLEFLGIQPT LGPPNQPPVS IWLIVFGVVM GVIVVGIVIL IFTGIRDRKK KNKARSGENP |
| 781 | YASIDISKGE NNPGFQNTDD VQTSE |

Coding region of mRNA (base 307 to 2721 of NM_021804.3) SEQ ID NO: 2

| | |
|---|---|
| 1 | atgtcaagct cttcctggct ccttctcagc cttgttgctg taactgctgc tcagtccacc |
| 61 | attgaggaac aggccaagac atttttggac aagtttaacc acgaagccga agacctgttc |
| 121 | tatcaaagtt cacttgcttc ttggaattat aacaccaata ttactgaaga gaatgtccaa |
| 181 | aacatgaata atgctgggga caaatggtct gcctttttaa aggaacagtc cacacttgcc |
| 241 | caaatgtatc cactacaaga aattcagaat ctcacagtca agcttcagct gcaggctctt |
| 301 | cagcaaaatg ggtcttcagt gctctcagaa gacaagagca aacggttgaa cacaattcta |
| 361 | aatacaatga gcaccatcta cagtactgga aaagtttgta acccagataa tccacaagaa |
| 421 | tgcttattac ttgaaccagg tttgaatgaa ataatggcaa acagtttaga ctacaatgag |
| 481 | aggctctggg cttgggaaag ctggagatct gaggtcggca agcagctgag gccattatat |
| 541 | gaagagtatg tggtcttgaa aaatgagatg gcaagagcaa atcattatga ggactatggg |
| 601 | gattattgga gaggagacta tgaagtaaat ggggtagatg gctatgacta cagccgcggc |
| 661 | cagttgattg aagatgtgga acataccttt gaagagatta aaccattata tgaacatctt |
| 721 | catgcctatg tgagggcaaa gttgatgaat gcctatcctt cctatatcag tccaattgga |
| 781 | tgcctccctg ctcatttgct tggtgatatg tggggtagat tttggacaaa tctgtactct |
| 841 | ttgacagttc cctttggaca gaaaccaaac atagatgtta ctgatgcaat ggtggaccag |
| 901 | gcctgggatg cacagagaat attcaaggag gccgagaagt tctttgtatc tgttggtctt |
| 961 | cctaatatga ctcaaggatt ctgggaaaat tccatgctaa cggacccagg aaatgttcag |
| 1021 | aaagcagtct gccatcccac agcttgggac ctggggaagg gcgacttcag gatccttatg |
| 1081 | tgcacaaagg tgacaatgga cgacttcctg acagctcatc atgagatggg gcatatccag |
| 1081 | tgcacaaagg tgacaatgga cgacttcctg acagctcatc atgagatggg gcatatccag |
| 1141 | tatgatatgg catatgctgc acaacctttt ctgctaagaa atggagctaa tgaaggattc |
| 1201 | catgaagctg ttggggaaat catgtcactt tctgcagcca cacctaagca tttaaaatcc |
| 1261 | attggtcttc tgtcacccga ttttcaagaa gacaatgaaa cagaaataaa cttcctgctc |
| 1321 | aaacaagcac tcacgattgt tgggactctg ccatttactt acatgttaga gaagtggagg |
| 1381 | tggatggtct ttaaagggga aattcccaaa gaccagtgga tgaaaaagtg gtgggagatg |
| 1441 | aagcgagaga tagttggggt ggtggaacct gtgccccatg atgaaacata ctgtgacccc |
| 1501 | gcatctctgt tccatgtttc taatgattac tcattcattc gatattacac aaggaccctt |
| 1561 | taccaattcc agtttcaaga agcactttgt caagcagcta aacatgaagg ccctctgcac |
| 1621 | aaatgtgaca tctcaaactc tacagaagct ggacagaaac tgttcaatat gctgaggctt |
| 1681 | ggaaaatcag aaccctggac cctagcattg gaaaatgttg taggagcaaa gaacatgaat |
| 1741 | gtaaggccac tgctcaacta ctttgagccc ttatttacct ggctgaaaga ccagaacaag |
| 1801 | aattcttttg tgggatggag taccgactgg agtccatatg cagaccaaag catcaaagtg |
| 1861 | aggataagcc taaaatcagc tcttggagat aaagcatatg aatggaacga caatgaaatg |
| 1921 | tacctgttcc gatcatctgt tgcatatgct atgaggcagt actttttaaa agtaaaaaat |
| 1981 | cagatgattc tttttgggga ggaggatgtg cgagtggcta atttgaaacc aagaatctcc |
| 2041 | tttaatttct ttgtcactgc acctaaaaat gtgtctgata tcattcctag aactgaagtt |
| 2101 | gaaaaggcca tcaggatgtc ccggagccgt atcaatgatg ctttccgtct gaatgacaac |
| 2161 | agcctagagt ttctggggat acagccaaca cttggacctc ctaaccagcc ccctgtttcc |
| 2221 | atatggctga ttgtttttgg agttgtgatg ggagtgatag tggttggcat tgtcatcctg |
| 2281 | atcttcactg ggatcagaga tcggaagaag aaaaataaag caagaagtgg agaaaatcct |
| 2341 | tatgcctcca tcgatattag caaaggagaa aataatccag gattccaaaa cactgatgat |
| 2401 | gttcagacct ccttt |

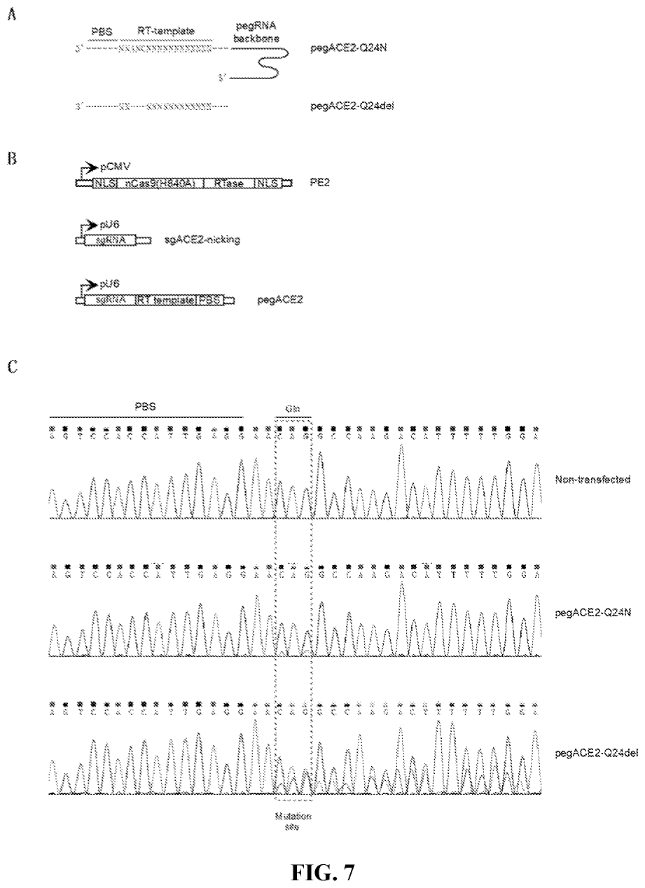

Also, pegACE2-Q24N|sgACE2-nicking and pegACE2-Q24del|sgACE2-nicking (FIG. 7A, 7B) can efficiently induced Q24N (C70A/G72C base substitutions in the coding region of ACE2 gene) and Q24del (C70Del/A71Del/G72Del base deletions in the coding region of ACE2 gene) mutations, respectively (FIG. 7C).

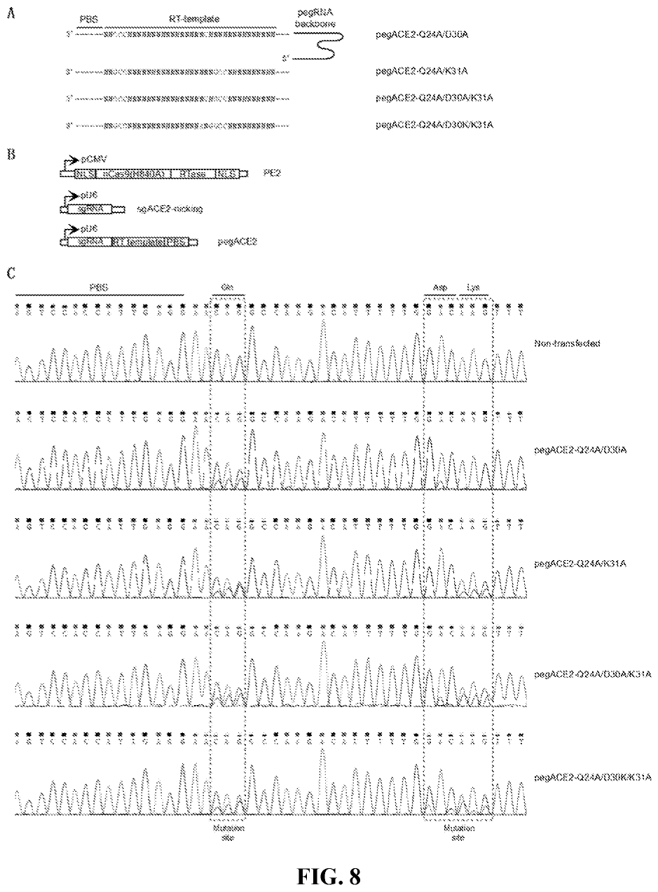

In addition, pegACE2-Q24A/D30A|sgACE2-nicking, pegACE2-Q24A/K31A|sgACE2-nicking, pegACE2-Q24A/D30A/K31A|sgACE2-nicking and pegACE2-Q24A/D30K/K31A|sgACE2-nicking (FIG. 8A, 8B) efficiently induced Q24A/D30A (C70G/A71C/G72C/A89C base substitutions in the coding region of ACE2 gene), Q24A/K31A (C70G/A71C/G72C/A91G/A92C/G93C base substitutions in the coding region of ACE2 gene), Q24A/D30A/K31A (C70G/A71C/G72C/A89C/A91G/A92C/G93C base substitutions in the coding region of ACE2 gene) and Q24A/D30K/K31A (C70G/A71C/G72C/G88A/C90G/A91G/A92C/G93C base substitutions in the coding region of ACE2 gene) mutations, respectively (FIG. 8C).

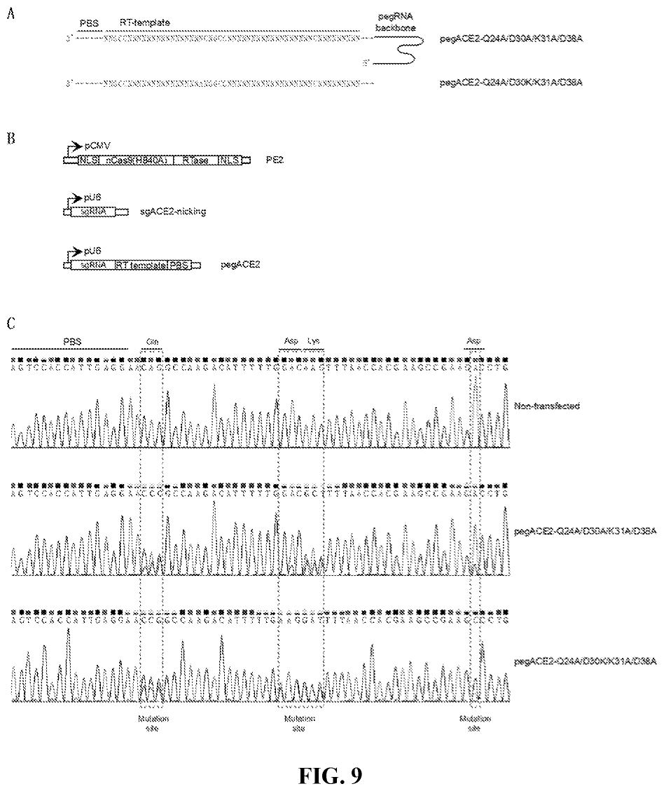

Then, pegACE2-Q24A/D30A/K31A/D38A|sgACE2-nicking and pegACE2-Q24A/D30K/K31A/D38A|sgACE2-nicking (FIG. 9A, 9B) efficiently induced Q24A/D30A/K31A/D38A (C70G/A71C/G72C/A89C/A91G/A92C/G93C/A113C base substitutions in the coding region of ACE2 gene) and Q24A/D30K/K31A/D38A (C70G/A71C/G72C/G88A/C90G/A91G/A92C/G93C/A113C base substitutions in the coding region of ACE2 gene) mutations, respectively (FIG. 9C).

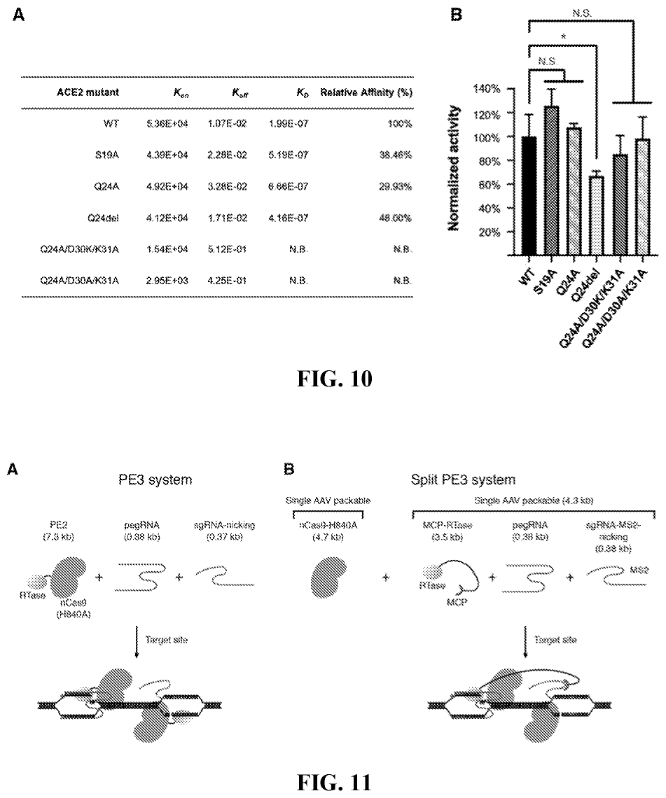

The corresponding ACE2 mutant proteins were purified and their binding affinity to the purified RBD of S protein was measured. It was found that the mutations S19A, Q24A, and Q24del all reduced the binding between ACE2 and RBD, while Q24A/D30A/K31A and Q24A/D30K/K31A abolished the binding (FIG. 10A). Furthermore, this example analyzed the angiotensin-converting activity of engineered ACE2 and found that the enzyme activities of S19A, Q24A, Q24A/D30A/K31A and Q24A/D30K/K31A were not significantly different from that of wild-type ACE2 though the mutations Q24del significantly reduced the enzyme activity (FIG. 10B).

Taken together, the engineered ACE2 proteins that contained the mutations Q24A/D30A/K31A and Q24A/D30K/K31A retained the angiotensin-converting activity but do not bind to the Spike protein of SARS-CoV-2. Thus, this example demonstrates a method to potentially prevent the infection of SARS-CoV-2 by using prime editor and the corresponding pegRNAs to induce mutations including Q24A/D30A/K31A and Q24A/D30K/K31A in human ACE2 gene.

The design of pegRNA and prime editing method described in this invention could be applied to perform high-efficiency base editing in the genome of various eukaryotes. The prime editing method and the editing product described in this invention could be applied to prevent or treat the infection of SARS-CoV-2.

Advantages of this invention: For the first time in the world, a prime editing system was established to achieve efficient editing by encoding silent mutations in pegRNA. The silent-mutation-encoding pegRNA method can be utilized to perform efficient single-base prime editing that cannot be implemented by the currently existing prime editing system at various genomic loci. Thus, the high efficiency of this new prime editing system will promote the potential clinical translation, especially in the gene therapies that are involved in restoring disease-related point mutations. Importantly, by introducing mutations in human ACE2 gene, the binding of S protein is abolished but the ACE2 catalytic activity is maintained, which can potentially be used to prevent COVID-19 infection or treat diseases caused by human coronaviruses.

### Example 4. PE-Mediated ACE2 Editing Enables Broad Prevention Against Multiple HCoVs

This example further expands and confirms the experiments of Example 3.

Despite current advances in pandemic control, emerging SARS-CoV-2 variants with extensive mutations in the spike protein raised new concerns for their enhanced transmissibility and potentials of immune escaping. Indeed, resistance to neutralization by antibodies, convalescent plasma or sera from vaccinated individuals has been consistently observed, challenging the efficacy of current vaccines and antibody therapeutics. Although equipped with a proofreading exonuclease nsp14, the error rate of viral genome replication is still high in coronaviruses (CoVs). It is thus foreseeable that SARS-CoV-2 will continue to accumulate mutations and new SARS-CoV-2 variants with fitness, transmission or immune escaping gains will continue to emerge, especially under selective pressures rendered by vaccination or drug treatment. Thus, the development of new interventions against SARS-CoV-2 is of importance, especially for interventions that would work once and for all.

As a constitutive trimer on the envelope of CoVs, the spike protein utilizes its S1 head piece to engage host receptors and its spring-loaded S2 stalk to drive the membrane fusion process. Hence, to block the entry of SARS-CoV-2 from the beginning, either the spike or its host receptor, i.e. angiotensin converting enzyme 2 (ACE2), can be targeted. Although soluble ACE2 (sACE2) have been exploited to serve as decoys for SARS-CoV-2, it is recently reported that sACE2 could surprisingly facilitate the cell entry of SARS-CoV-2 through receptor-mediated endocytosis, raising concerns for the decoy strategy. Here, we opt for an alternative strategy to precisely reshape the host ACE2 in situ, taking advantage of the most advanced genome editing tool.

This example sought to use prime editor to perform structure-guided editing of human ACE2 (hACE2), aiming to preserve the physiological function of hACE2 in renin-angiotensin system (RAS) while ablating its role as the SARS-CoV-2 receptor. The results indicate that PE-mediated editing of hACE2 enables broad prevention against multiple human coronaviruses (HCoVs) including various SARS-CoV-2 strains, SARS-CoV and HCoV-NL63.

### Materials and Methods

### Plasmids Construction

Primer sets (hACE2_PCR_F/hACE2_PCR_R) were used to amplify the full-length wildtype Human ACE2 (hereafter referred as hACE2) gene from pUC57-Human_ACE2 template (synthesized by Genscript). The amplified gene fragment was then cloned into the pcDNA3.1_pCMV-HA vector using ClonExpress® II One Step Cloning kit (Vazyme, C112-02) to generate the hACE2 expression plasmid pcDNA3.1_pCMV-hACE2-HA. The expression plasmids of different hACE2 variants, including pcDNA3.1_pCMV-hACE2_AAA-HA, pcDNA3.1_pCMV-hACE2_AKA-HA, pcDNA3.1_pCMV-hACE2_K353del-HA, pcDNA3.1_pCMV-hACE2_AAA/K353del-HA and pcDNA3.1_pCMV-hACE2_AKA/K353del-HA, were all constructed through site-directed mutagenesis and verified by DNA sequencing. Two primer sets (U6_peg_F/U6_Q24_R) (U6_Q24A_F/U6_Q24A_R1/R2) were used to amplify the U6-peg-Q24A fragment. Then, the fragment was cloned into the pGL3-U6-sgRNA-PGK-puromycin vector to generate the pegQ24A expression plasmid pGL3-U6-pegQ24A. Other pegRNA expression plasmids were constructed in similar ways. Oligonucleotides hACE2_nick_FOR/hACE2_nick_REV were annealed and ligated into BsaI linearized pGL3-U6-sgRNA-PGK-puromycin vector to generate the sg_nickRNA expression plasmids psgnick_ACE2. Other sg_nickRNA expression plasmids were constructed in similar ways.

The extracellular protease domain (PD) of ACE2 (residues 19-615, hereafter referred to as hACE2-PD), the receptor binding domain (RBD) of wildtype, B.1.1.7, B.1.351 or P1 SARS-CoV-2 (residues 319-541), as well as the RBD of SARS-CoV (residues 306-527) were each cloned into a modified pFastBac vector with an N-terminal gp67 signaling peptide and a C-terminal thrombin cleavage site followed by a strep tag and a 6× His tag. The RBD of HCoV-NL63 (residues 481-616) was fused with an with an N-terminal gp67 signaling peptide and a C-terminal PreScission Protease (PSP) cleavage site followed by a 6× His tag, and cloned into a pFastBac vector. All other hACE2-PD variants were then constructed through site-directed mutagenesis and verified by DNA sequencing.

### Protein Expression and Purification

Recombinant bacmids were prepared using the BacToBac system (Invitrogen) following manufacturer's manual. Baculoviruses were generated by transfecting Sf9 cells at 70% confluency with freshly prepared bacmids using FuGene HD (Promega). After 72 hrs, the medium of transfected cells were harvested and used as P1 virus stocks. P2 viruses were generated from P1 stocks at low MOI, supplemented with 1% FBS and stored at 4° C. in the dark until further use.

For expression of the hACE2-PD, 1 L HighFive cells at 2×10⁶ cells/ml were infected with 20 mL P2 virus and harvested at 60 h by centrifugation at 5000 g for 10 min. The supernatant was then supplemented with 1 mM phenylmethanesulfonyl fluoride (PMSF) and loaded onto an Excel Ni-NTA column (GE Healthcare) equilibrated with buffer A (50 mM Tris-HCl, pH 8.0, 250 mM NaCl). The column was then washed with buffer A supplemented with 5 mM imidazole before being eluted with a linear gradient of 5-500 mM imidazole. After SDS-PAGE examination, elution fractions containing the ACE2-PD protein were pooled and further purified using size exclusion chromatography (SEC) in buffer B (20 mM Tris-HCl, pH 8.0, 150 mM NaCl) with a Superdex S200 column (GE Healthcare). SEC fractions containing hACE2-PD were then pooled, concentrated and stored at −80° C. until further use. Other hACE2-PD variants and different RBD proteins were expressed and purified use similar methods as described before.

### Cell Culture and Transfection

HEK293FT cells from ATCC were maintained in DMEM (10566, Gibco/Thermo Fisher Scientific) supplemented with 10% FBS (16000-044, Gibco/Thermo Fisher Scientific). The cells have been tested to exclude mycoplasma contamination.

For generation of hACE2-KO 293FT stable cell lines, HEK293FT cells were seeded in a 6-well plate at a density of 3×10⁵ per well and transfected with 200 μl serum-free Opti-MEM that contained 4.92 μl LIPOFECTAMINE LTX (Life, Invitrogen), 1.64 μl LIPOFECTAMINE plus (Life, Invitrogen), 1 μg Cas9 expression vector pCMV-Cas9 and 0.64 μg sgRNA_ko expression plasmid. After 72 h, the transfected cells were seeded in 96-well plate at a density of 1 per well. And After 3-4 weeks, the genomic DNA was extracted from the cells with QuickExtract™ DNA Extraction Solution (QE09050, Epicentre) or the cells were lysed in 2×SDS loading buffer for western blot.

For construction cell lines that exogenously over-expresses hACE2, hACE2-KO 293FT cells were seeded in a 6-well plate at a density of 3×10⁵ per well and transfected with 200 μl serum-free Opti-MEM that contained 4.5 μl LIPOFECTAMINE LTX (Life, Invitrogen), 1.5 μl hACE2 expression plasmid (or hACE2-AAA, hACE2-AKA, hACE2-K353del, hACE2-AAA/K353del or hACE2-AKA/K353del expression plasmid). After 72 h, the cells were lysed in 2χ SDS loading buffer and subjected to western blot.

For generation of EF1αP-KI 293FT stable cell lines wherein the expression of endogenous hACE2 was enhanced under the control of introduced EF1α promoter, HEK293FT cells were seeded in a 6-well plate at a density of 3×10⁵ per well and transfected with 200 μl serum-free Opti-MEM that contained 4.92 μl LIPOFECTAMINE LTX (Life, Invitrogen), 1.64 μl LIPOFECTAMINE plus (Life, Invitrogen), 1 μg Cas9 expression vector pCMV-Cas9 and 0.64 μg sgRNA_kin expression plasmid. After 72 h, the transfected cells were seeded in 96-well plate at a density of 1 per well. And After 3-4 weeks, the genomic DNA was extracted from the cells with QuickExtract™ DNA Extraction Solution (QE09050, Epicentre) or the cells were lysed in 2×SDS loading buffer for western blot.

For Prime editing of endogenous hACE2 gene, EF1aP-KI 293FT cells were seeded in a 24-well plate at a density of 1.6×10⁵ per well and transfected with 200 μl serum-free Opti-MEM that contained 4.92 μl LIPOFECTAMINE LTX (Life, Invitrogen), 1.64 μl LIPOFECTAMINE plus (Life, Invitrogen), 1 μg PE2 expression vector pCMV-PE2 (addgene, 132775), 0.44 μg pegRNA expression plasmid and 0.2 μg sgnickRNA expression plasmid. After 72 h, the transfected cells with QuickExtract™ DNA Extraction Solution (QE09050, Epicentre).

### Antibodies

Antibodies were purchased from the following sources: β-Actin Mouse Monoclonal Antibody (Absci, AB21800); Rabbit monoclonal [EPR4435(2)] to ACE2 (Abcam, ab108252); ACE2 Recombinant Rabbit Monoclonal Antibody [SN0754] (Thermo Fisher Scientific, MA532307); Donkey polyclonal Secondary Antibody to Rabbit IgG—H&L (Alexa Fluor® 647, Abcam, ab150075); PE Anti-6× His Tag® antibody (AD.1.1.10) (Abcam, ab72467).

### Western Blot

Transfected cells were lysed in NP40 lysis buffer (50 mM Tris pH 8.0, 150 mM NaCl, 1% NP-40, 0.1% SDS, 1 mM PMSF, protease inhibitors, and phosphatase inhibitor) for 30 min on ice, then incubated at 97° C. for 15 min, separated by SDS-PAGE (Genscript) in sample loading buffer and proteins were transferred to nitrocellulose membranes (Thermo Fisher Scientific). After blocking with TBST (25 mM Tris pH 8.0, 150 mM NaCl, and 0.1% Tween 20) containing 5% (w/v) nonfat dry milk and 1% BSA for 2 h, the membrane was reacted overnight with indicated primary antibody. After extensive washing, the membranes were reacted with HRP-conjugated secondary antibodies for 1 h. Reactive bands were developed in ECL (Thermo Fisher Scientific) and detected with Amersham Imager 680.

### Angiotensin II Converting Enzymatic Assay

For recombinant hACE2-PD or its variants, the enzymatic assay was performed using the ACE2 Protease Activity Assay kit (Biovision). Briefly, different concentrations of substrate were prepared in a 96-well plate with a total reaction volume of 100 μl at 25° C. The reactions were initiated by the addition of hACE2-PD or its variants at a final concentration of 1 μM, and the fluorescence signals were measured (Ex/Em 320 nm/420 nm) in a kinetic mode on MD-SpectraMax i3 (Molecular Devices). The data were analyzed by Michaelis-Menten curve fitting with Origin software (OriginLab).

For enzymatic analysis of cell lysates, transfected cells were lysed with Lysis Buffer and assayed according to the manual of Angiotensin II Converting Enzyme (ACE2) Activity Assay Kit (AssayGenie, #BN01071). Protein concentration in the lysate were measured by BCA Protein Assay Kit (YEASEN, 20202ES76). Fluorescence data was measured with MD-SpectraMax i3 (Molecular Devices) and fitted as described above.

### Surface Plasmon Resonance (SPR) Assay

SPR experiments were performed using a Biacore 8K instruments (GE Healthcare). All assays were performed with a running buffer containing 10 mM HEPES pH 7.4, 150 mM NaCl, 3 mM EDTA and 0.01% v/v Surfactant P20 at 25° C. For affinity determinations, recombinant RBD proteins of wildtype, B.1.1.7 RBD, B.1.351 or P.1 SARS-CoV-2 strains and that of HCoV-NL63 or SARS-CoV were each immobilized to a single flow cell on CM5 sensor chips (GE Healthcare). Three samples containing only running buffer were injected over both ligand and reference flow cells, followed by serial dilutions of recombinant hACE2-PD or its variants from 200 nM to 3.125 nM. All the binding data were double referenced by blank cycle and reference flow cell subtraction. The resulting sensorgrams were fit to a 1:1 Langmuir binding model using the Biacore Insight Evaluation Software (GE Healthcare).

Homology Model Building of Selected hACE2 Mutants

Models of the hACE2-PD variants, namely AKA (Q24A/D30K/K31A), AAA (Q24A/D30A/K31A), K353del, AKA/K353del and AAA/K353del, were derived by homology modeling using Swiss Model website. Initial models were optimized by performing energy minimization followed by 5 ns molecular dynamics simulation using Schrödinger Suite 2019-1 (https://www.schrodinger.com). The simulation systems were solvated with full atom TIP3P water, containing Cl⁻ and Na⁺ ions at a concentration of 0.15 M to mimic physiological ionic strength. During the simulation, Temperature T and pressure P were kept constant, at 310 K and 1 atm respectively.

### Flowcytometry

The RBD from wildtype, B.1.1.7, B.1.351 or P1 SARS-CoV-2 was incubated at a concentration of 20 μg/mL with 1*10⁶ 293FT cells that exogenously over-express different hACE2 variants in 500 μl PBS for 60 mins at room temperature. After washing twice with PBS containing 2% FBS (16000-044, Gibco/Thermo Fisher Scientific). Cell were resuspended and incubated with anti-His tag antibody with PE (Abcam) for 30 mins at 4° C. in dark. Cells were then washed for three times and resuspended with PBS containing 2% FBS before being analyzed using CytoFLEX (Beckman Coulter). ACE2-Knockout 293FT cells were used as negative controls. Data were analyzed using Flowjo software.

### Analysis of SARS-CoV-2 Pseudovirus Entry

Pseudotyped SARS-CoV-2 viruses were purchased from the following sources: SARS-CoV-2-Fluc WT (Vazyme, DD1402-03); SARS-CoV-2-Fluc B.1.1.7 (Vazyme, DD1440-03); SARS-CoV-2-Fluc 501Y.V2 (Vazyme, DD1441-03); SARS-CoV-2-Fluc P.1 (Vazyme, DD1446-03). The pseudoviruses were then used to infect 293T cells (104 per well in 96-well plates) that exogeneously or endogenously overexpress hACE2 or its variants. After 48 h, infected cells were washed with PBS, lysed and analyzed for intracellular luciferase activity with the Luciferase Reporter Gene Assay Kit (YEASEN, 11401ES60*) according to manufacturer's instructions. Luminescence was recorded on a Tecan-Spark (Tecan).

### DNA Library Preparation and Sequencing

Target genomic sites were PCR amplified by high-fidelity DNA polymerase PrimeSTAR HS (Clonetech) with primers flanking each examined sgRNA target site. Indexed DNA libraries were prepared by using the TruSeq ChIP Sample Preparation Kit (Illumina) with some minor modifications. Briefly, the PCR products amplified from genomic DNA regions were fragmented by Covaris S220. The fragmented DNAs were then PCR amplified by using the TruSeq ChIP Sample Preparation Kit (Illumina). After being quantitated with Qubit High-Sensitivity DNA kit (Life, Invitrogen), PCR products with different tags were pooled together for deep sequencing by using the Illumina Hiseq 2500 (1×100) or Hiseq X-10 (2×150) at CAS-MPG Partner Institute for Computational Biology Omics Core, Shanghai, China. Raw read qualities were evaluated by FastQC (www.bioinformatics.babraham.ac.uk/projects/fastqc/). For paired-end sequencing, only R1 reads were used. Adaptor sequences and read sequences on both ends with Phred quality score lower than 28 were trimmed. Trimmed reads were then mapped with the BWA-MEM algorithm (BWA v0.7.9a) to target sequences. After being piped up with samtools (v0.1.18), indels and base substitutions were further calculated.

### Amino Acid Substitution Calculation

Amino acid substitutions were selected at each position of the examined pegRNA target sites that mapped with at least 1,000 independent reads, and obvious amino acid substitutions were only observed at the targeted editing sites. Amino acid substitution frequencies were calculated by dividing base substitution reads by total reads.

### Statistical Analysis

P values were calculated from two-tail Student's t test in this study.

### Results

Structure-Guided Genome Editing of hACE2

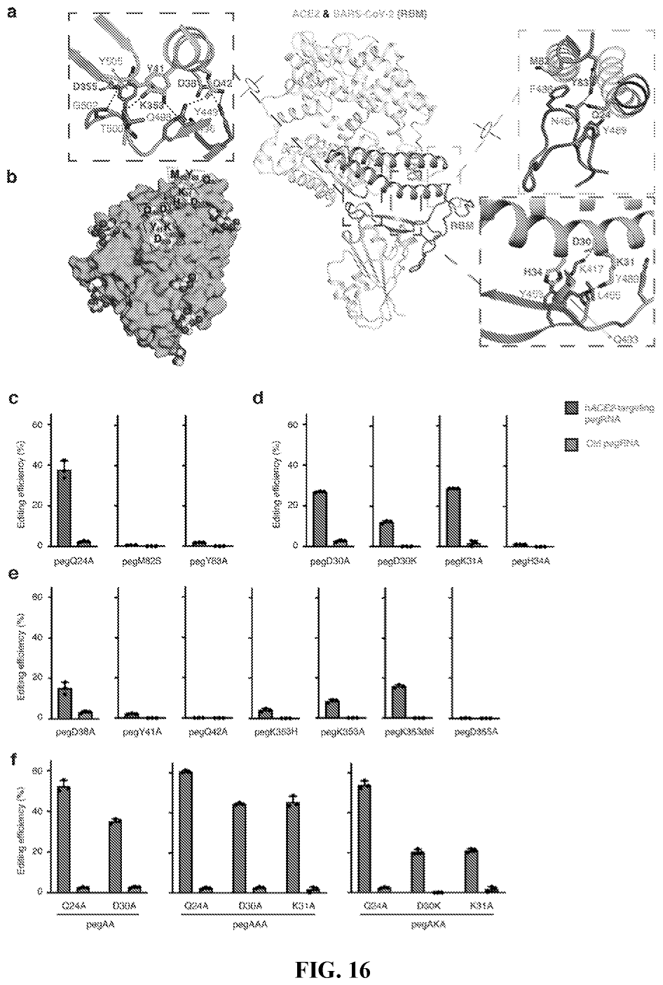

Akin to SARS-CoV, SARS-CoV-2 also utilizes the receptor binding domain (RBD) from its S1 subunit of spike protein to engage the host receptor ACE2. When choosing editing sites, we mainly took into consideration the structure of full-length hACE2 in complex with SARS-CoV-2 RBD, as well as that of intact SARS-CoV-2 spike in complex with hACE2 peptidase domain, in order to eliminate serendipity and identify consensus residues that are most determinant for the interaction between hACE2 and spike. The interface between the two mainly involves the receptor binding motif (RBM) in SARS-CoV-2 RBD (FIG. 16a, salmon), as well as the α1-α2 helices and β3-β4 loop on hACE2 (FIG. 16a, cyan), wherein interface residues of hACE2 could be further grouped into three clusters (FIGS. 16a and 16b, dashed boxes). Consensus residues that interact with the so-called receptor binding ridge of RBD mainly involves residues Q24, M82 and Y83 on hACE2 (FIG. 16a, b, orange box, cluster 1), while residues D30, K31 and H34 on hACE2 constitutes the major RBD interacting sites in the middle of the interface (FIGS. 16a and b, red box, cluster 2). At the other end of the interface, D38, Y41, Q42 from al and K353, D355 from the β3-β4 loop of hACE2 are the consensus residues chosen for editing (FIG. 16a, b, purple box, cluster 3).

We then designed prime editing guide RNAs (pegRNAs) to change the ACE2 residues mentioned above to the ones that disfavor the interaction to SARS-CoV-2 RBD (FIG. 16c-f). By using PE3 system, we first changed these residues one by one and found that the editing efficiencies were generally low (FIG. 16c-e, e.g., lower than 30%, except for pegQ24A). But when changing multiple resides in clusters 1 and 2 simultaneously, we found that the editing efficiencies increased (comparing FIG. 16f with FIG. 16c-e), particularly for pegAAA (designed for Q24A/D30A/K31A) and pegAKA (designed for Q24A/D30K/K31A), both of which edit Q24 in cluster 1 and D30 and K31 in cluster 2 (FIG. 16a, b). For the residues in cluster 3, though the pegRNAs for residue changes induced relatively low editing efficiency, pegK353del that was designed to delete the codon for K353 induced ˜20% editing efficiency (FIG. 16e). According to the editing efficiencies at tested sites (FIG. 16c-f), pegAA, pegAAA and pegAKA induced efficient genome editing (maximal efficiency >30%) for cluster 1 and 2 residue change and pegK353del induced relatively efficient editing (˜20% efficiency) for cluster 3 residue deletion. Thus, these sites and their combinations were chosen for subsequent biochemical evaluation.

Mutated hACE2 No Longer Binds SARS-CoV-2 RBDs

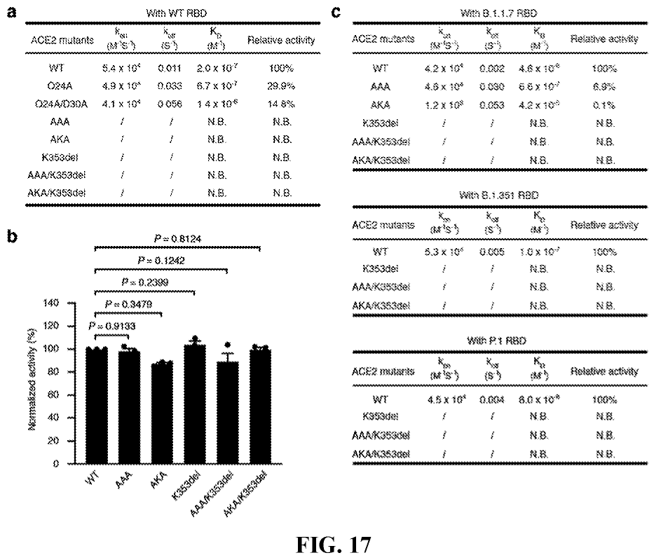

To understand the possible outcomes of hACE2 editing, we expressed and purified the extracellular protease domain of wildtype (WT) hACE2 (WT hACE2ecd) and its editing-derived mutants, and characterized their binding kinetics and affinity to WT SARS-CoV-2 RBD (WT RBD) via surface plasmon resonance (SPR). The WT hACE2ecd binds to immobilized WT RBD with equilibrium disassociation constant (K_{D}) of 200 nM, while hACE2ecd mutants Q24A and Q24A/D30A bind to WT RBD with K_{D} of 670 nM and 1.4 μM respectively, each representing a 70.1% and 85.2% reduction in affinity (FIG. 17a). To our surprise, the hACE2ecd mutants AAA, AKA, K353del, AAA/K353del and AKA/K353del cannot bind WT RBD at all (FIG. 17a), suggesting that corresponding editing at these sites might prevent hACE2 to serve as the receptor for SARS-CoV-2.

Besides acting as the entry point for several CoVs, the primary physiological function of ACE2 is to catalyze the conversion of angiotensin (Ang) II, a vasoconstrictor, into angiotensin, a vasodilator, thereby counteracting the activity of Angiotensin-converting enzyme (ACE) to keep the blood pressure and fluid or electrolyte balance. Hence, when editing hACE2 to prevent hACE2 being hijacked by CoVs as entry point, it is of great importance not to disturb its Ang II hydrolysis function. We thus measured the catalytic activity of WT hACE2ecd and its five mutants which no longer binds SARS-CoV-2 RBD (FIG. 17b). The results indicate that the enzyme activity of hACE2 was not affected at all by these mutations (FIG. 17b), consistent with the observation that hACE2 mutants manifest no major conformational changes in its protease domain as compared to WT hACE2.

Since its initial emergence at the end of 2019, SARS-CoV-2 has undergone considerable evolution. At early stage, the genome of SARS-CoV-2 remained relatively stable, except for a D614G substitution in the viral spike protein that quickly became dominant in global circulating strains. Starting from the latter half of 2020, the evolution of SARS-CoV-2 is considered to be accelerated, and several SARS-CoV-2 strains with increased transmissibility and harboring immune escaping potentials emerged successively in different regions of the world, first in U.K. (B.1.1.7), and then in South Africa (B.1.351) and Brazil (P.1). These strains all contain mutations in the RBD of spike (B.1.1.7: N501Y; B.1.351: K417N/E484K/N501Y; P.1: K417T/E484K/N501Y), raising the possibility of altered hACE2 interaction mode.

To test if the editing of hACE2 at previously identified sites would still prevent hACE2 to be bound by these SARS-CoV-2 strains, we also investigated the binding kinetics and affinity of WT or mutant hACE2ecd to the RBDs of these strains (FIG. 17c). The WT hACE2ecd binds to immobilized B.1.1.7 RBD with K_{D} of 46 nM, suggesting that WT hACE2ecd might has a higher affinity for B.1.1.7 RBD than for WT RBD. Of the five hACE2ecd mutants which no longer binds WT RBD, mutants AAA and AKA binds B.1.1.7 RBD with K_{D} of 660 μM and 42 nM respectively, while mutants K353del, AAA/K353del and AKA/K353del did not show any binding to B.1.1.7 RBD (FIG. 17b, top panel). These results indicate that mutants K353del, AAA/K353del and AKA/K353del may harbor broad resistance to the binding of RBDs from different SARS-CoV-2 strains. Indeed, although the WT hACE2ecd binds immobilized RBDs from B.1.351 and P.1 with K_{D} of 100 nM and 80 nM respectively, essentially no binding was observed between the above three mutants and these two RBDs (FIG. 17c, middle and bottom panels).

From the above biochemical data, hACE2ecd mutants K353del, AAA/K353del and AKA/K353del are broadly resistant to the binding of RBDs from all tested prevalent SARS-CoV-2 strains, prompting us to determine if full-length hACE2 proteins harboring the above residue changes are resistant to these prevalent RBDs at the cell surface.

PE-Mediated hACE2 Editing Enables Broad Prevention Against Different SARS-CoV-2 Strains

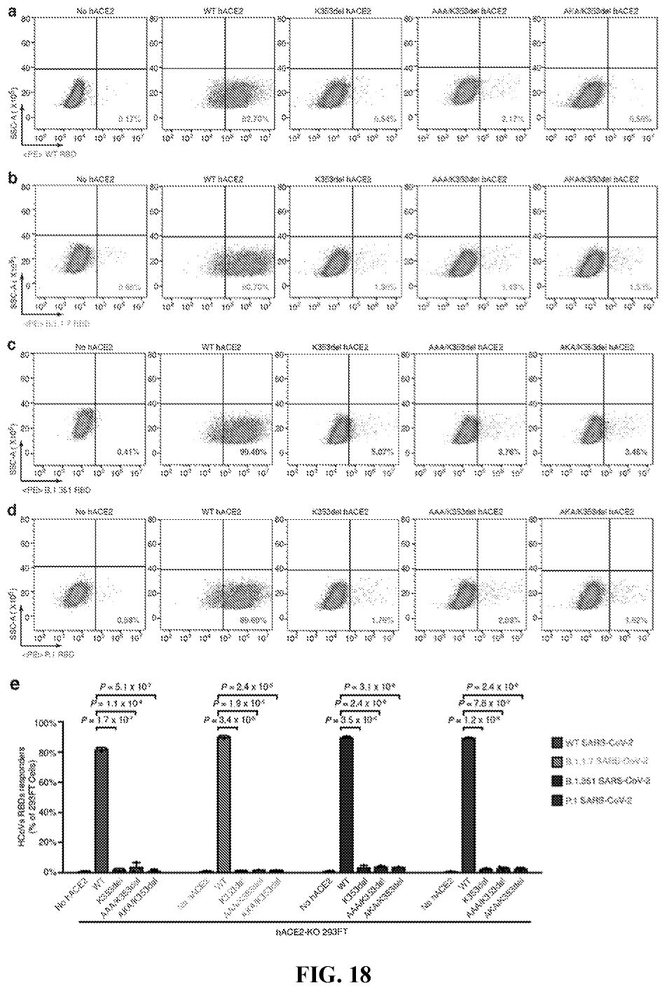

To evaluate the binding capacity of different SARS-CoV-2 RBDs to full-length hACE2 or its mutants through a flow-cytometry-based assay, we first established a 293FT cell line wherein the hACE2 was knocked out (hereafter referred as hACE2-KO 293FT cells) with Cas9 nuclease and the expression of endogenous hACE2 was eliminated. We next exogenously over-expressed WT hACE2 or its mutants K353del, AKA/K353del and AAA/K353del in hACE2-KO 293FT cells and the ACE2 protein expression levels and Ang II converting activities were similar. The flow cytometry data showed that the RBDs from all tested SARS-CoV-2 strains could bind to the WT-hACE2-expressing cells efficiently, with 81.7±1.3%, 89.9±0.9%, 89.6±0.7% and 89.2±0.4% cells stained positive for WT RBD, B.1.1.1 RBD, B.1.351 RBD and P.1 RBD respectively (FIG. 18a-d, the left second panels and FIG. 18e). In contrast, the RBDs from all tested SARS-CoV-2 strains bind to K353del, AAA/K353del or AKA/K353del-expressing cells with drastically lower positive rates, with only 1.8±1.1%, 1.3±0.85% or 3.6±3.2% of these cells stained positive for WT RBDs, 1.4±0.06%, 1.6±0.15% or 1.6±0.19% cells stained positive for B.1.1.7 RBDs, 3.5±1.4%, 3.5±0.23% or 4.0±0.50% cells stained positive for B.1.351 RBDs and 2.3±0.51%, 2.6±0.87% or 2.9±0.75% cells stained positive for P.1 RBDs, all of which are close to the responding rates of hACE2-KO controls (FIG. 18a-d, left third to fifth panels comparing to left first two panels and FIG. 18e).

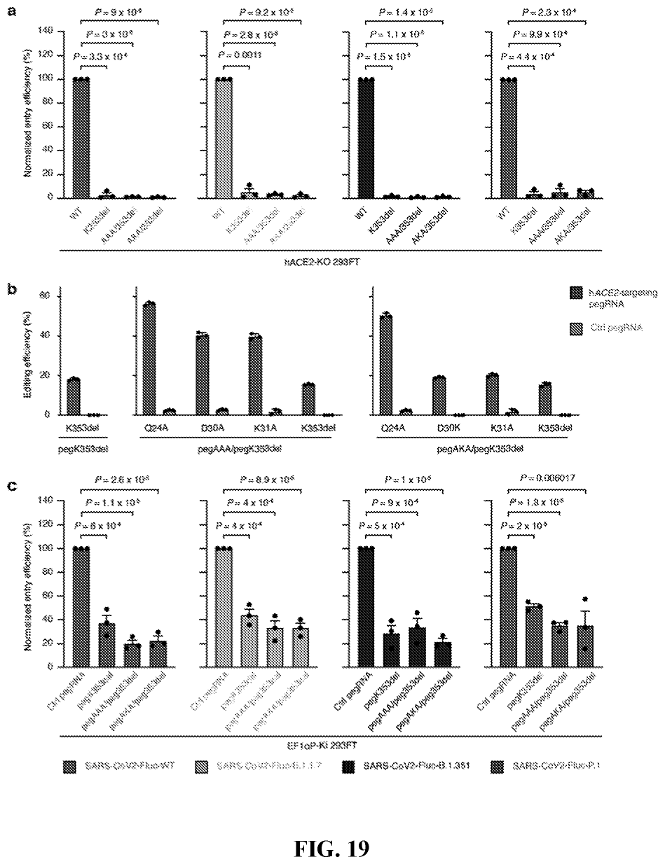

Next, we sought to interrogate the resistance of these K353del, AAA/K353del or AKA/K353del-expressing cells to SARS-CoV-2 infection through pseudovirus experiments. Pseudotyped viruses carrying spike proteins from different SARS-CoV-2 strains were used to infect hACE2-KO 293FT cells that exogenously overexpress WT hACE2 or its editing-derived mutants. The results indicated that cells expressing hACE2 K353del, AAA/K353del or AKA/K353del mutants were generally resistant to infections of all tested SARS-CoV-2 strains (FIG. 19a).

Although cells expressing the aforementioned hACE2 mutants were resistant to SARS-CoV-2 infection, we further tried to edit the endogenous hACE2 with pegK353del, pegAAA and pegAKA in PE3 system to examine whether bona fide genome editing of hACE2 can confer edited cells resistance to SARS-CoV-2 variants. As the expression of hACE2 in 293FT cells is low, we knocked in a EF1α promoter upstream to the transcription start site of hACE2 to boost the expression of endogenous hACE2. We then edited the EF1α-promoter-knockin (EF1αP-KI) 293FT cells by using only pegK353del and pegK353del with pegAAA or pegAKA in PE3 system and found that pegK353del, pegAAA/pegK353del and pegAKA/pegK353del all induced efficient editing at on-target sites and no observable mutation at predicted off-target sites [PMID: 24463181]. The expression hACE2 proteins and their enzyme activities were not affected. Next, the edited EF1αP-KI cells were infected with pseudotyped viruses carrying the spike proteins from different SARS-CoV-2 strains. Compared with control-pegRNA-edited cells, cells that were edited with pegK353del, pegAAA/pegK353del or pegAKA/pegK353del were broadly resistant to the infection of all tested SARS-CoV-2 strains with similar levels of resistance (FIG. 19c), indicating that PE-mediated genome editing of hACE2 realized broad prevention against SARS-CoV-2 strains.

hACE2 Mutants are Also Resistant to SARS-CoV and HCoV-NL63

Besides SARS-CoV-2, SARS-CoV and HCoV-NL63 also use hACE2 as their host receptors. Interestingly, these HCoVs seems to exploit overlapping surface areas on hACE2 for their landing. Of all the residues on this face of hACE2, which makes direct contact with three different HCoVs, interface residues H34, Y41 and K353 are shared among the three HCoVs. Although the editing of H34 and Y41 was not efficient, the editing of K353 was successfully achieved (FIG. 19b) and manifested broad-spectrum blocking effect to the infections of various SARS-CoV-2 strains (FIG. 19c). These results prompt us to wonder whether these editing products would exhibit even broader anti-HCoVs effects against SARS-CoV and NL63.

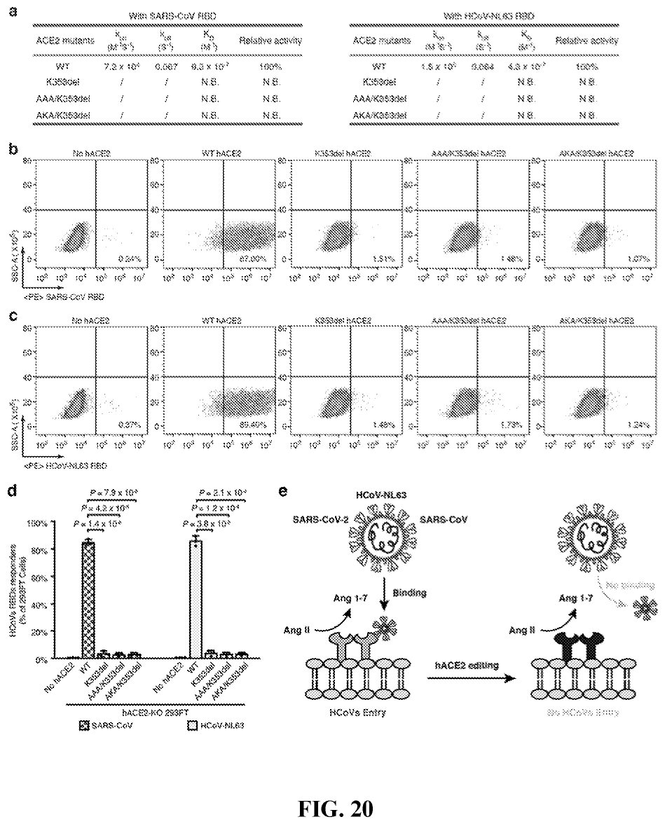

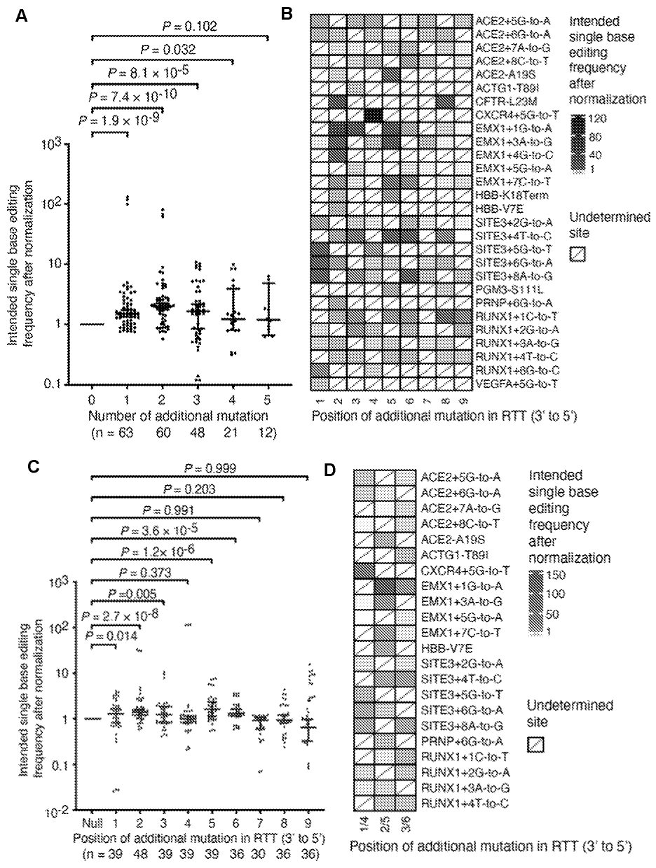

SPR results showed that hACE2ecd isoforms with K353del, AAA/K353del or AKA/K353del mutations no longer bind the RBDs from SARS-CoV and HCoV-NL63 at all (FIG. 20a). Consistently, 293FT cells expressing full-length hACE2 with these mutations were not responsive to the RBDs from SARS-CoV and HCoV-NL63 either (FIG. 20b-d), suggesting that editing of hACE2 at corresponding sites would prevent hACE2 to serve as the host receptor for SARS-CoV and HCoV-NL63 as well.

In this example, we used the latest genome editing tool, PE, to engineer hACE2, aiming to reshape its extracellular regions for ablating its role as SARS-CoV-2 receptor. By taking advantage of the high editing precision and efficiency of PE, hACE2 residues involved in the interaction with various spike proteins were changed or deleted accurately, which blocks the binding and entry of all tested hCoVs, including four globally prevalent SARS-CoV-2 variants, SARS-CoV and NL63, while maintains the Ang II hydrolysis activity (FIG. 20e).

From these results, some residues (e.g., K353) are conservative for the binding of many hCoVs and the precise editing of these residues confers broad-spectrum prevention against the type of hCoVs that uses hACE2 as their host receptor. As new SARS-CoV-2 variants with enhanced ability of transmission or immune escaping, e.g., variant B.1.617 that is becoming the major strain in India and south Asia, are and will be emerging, the genome editing way in this study provides a proof of concept for the broad prevention of to-appear SARS-CoV-2 variants. Reminiscently, editing the host receptor for another epidemic-causing virus, i.e. C—C chemokine receptor type 5 (CCR5) for HIV, is being actively investigated in clinic trials [PMID: 31509667]. As a highly precise and efficient genome editing tool, we demonstrated the potential that PE can be applied to treat or prevent epidemic human diseases and it is envisioned that this versatile genome editing tool will be employed in treating more types of diseases in the future.

Interestingly, we noticed that modest genome editing rates of hACE2 provided unexpectedly high efficiency to suppress the entry of SARS-CoV-2 variants (comparing panels b and c of FIG. 19). This phenomenon implied that the complete invasion of SARS-CoV-2 may require a high concentration of WT hACE2 on cell membrane, which awaits further investigation.

Though hACE2 is considered the major receptor for SARS-CoV-2, other membrane proteins have been reported to be as potential host receptors that are involved in the host entry of SARS-CoV-2. The data in this study showed that the change or deletion of a few key residues of hACE2 can very efficiently block the invasion of SARS-CoV-2 variants (FIG. 19a,c), indicating that hACE2 is indispensable for the host entry and infection of SARS-CoV-2. Among tested resides, the deletion of only K353 can block the entry of all hCoVs we examined (FIG. 19a,c), suggesting that this residue may be a potential target for component screening to treat COVID-19.

### Example 5. Split Prime Editing

A complete prime editor PE3 requires a construct (about 11 kb) that is much larger than what an AAV vehicle can accommodate. Accordingly, a Split PE3 system was designed and tested. The original PE3 system is illustrated on the left panel of FIG. 11A, and the newly designed Split PE3 system is illustrated in FIG. 11B, in which constructs encoding the nickase and the RTase are packaged into different AAV particles. The RTase is fused to an RNA binding protein MCP, and the sgRNA-nicking includes a binding site MS2. When taken up into a cell, the RTase can be recruited by the sgRNA-nicking, through the MS2-MCP binding, and come in contact with the nickase.

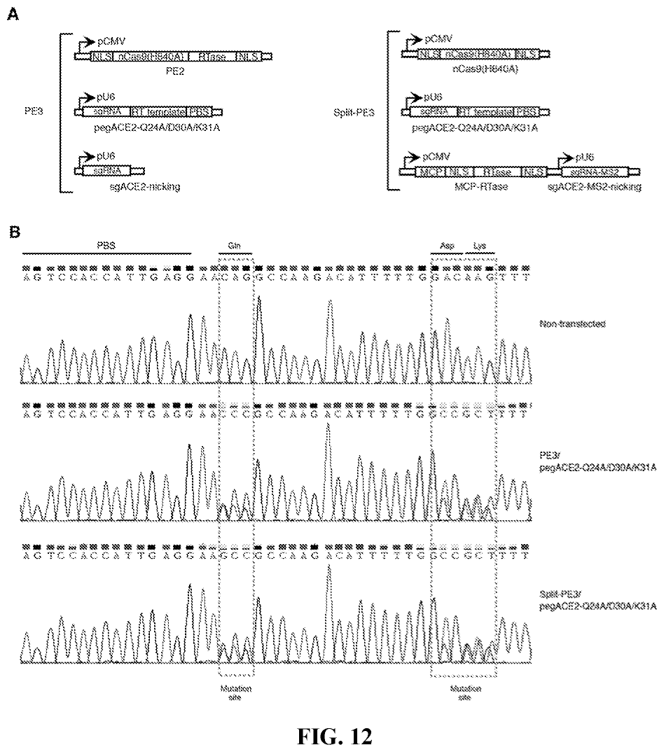

The tested constructs are illustrated in FIG. 12A (left: original PE3; right: split PE3) targeting the ACE2 gene (Q24del). The first construct included the coding sequence for nCas(H840A), which had a size suitable for packaging into an AAV. The other two separate constructs had a total size that is also suitable for packaging into an AAV. One construct encoded the pegRNA and the other encoded the RTase and the sgRNA. As shown in the results (FIG. 12B), both configurations produced excellent results.

In the experiment demonstrated in FIG. 13A-B, the split PE3 system was tested with three mutations (Q24A/D30A/K31A) in the ACE2 gene. As shown, the editing efficiencies of both the original PE3 and the split PE3 were excellent, and were higher than the experiment as shown in FIG. 12A-B.

### Example 6. Multiple Substitutions

This example analyzed the relationship between additional base substitution numbers and editing efficiency.

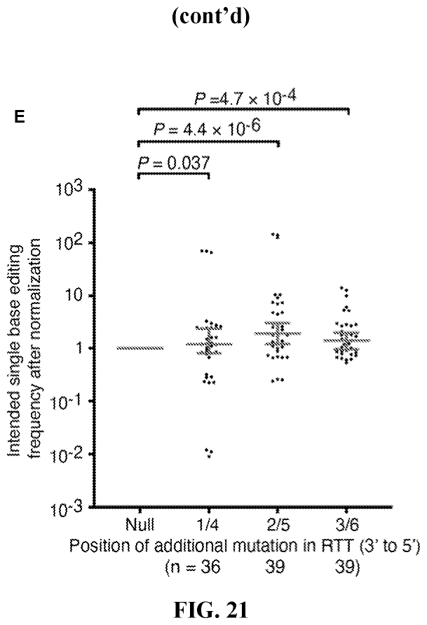

It's discovered that introducing no more than four additional base substitutions in reverse transcription-template (RTT) could significantly improve the editing efficiency (FIG. 21A) and that introducing two additional base substitutions induced the highest efficiencies (P=7.4×10⁻¹⁰, FIG. 21A). After examining the data from all 114 single additional base substitution-containing pegRNAs across thirteen target sites (FIG. 21), this example found that the introduction of a single additional base substitution at positions 1, 2, 3, 5 and 6 significantly improved the intended base editing efficiency (median 1.28-, 1.41-, 1.23-, 1.62- and 1.32-fold, respectively, FIG. 21C).

As adding two additional base substitutions induced the highest editing efficiencies (FIG. 21A), this example then introduced double additional base substitutions at positions 1/4, 2/5 and 3/6, which were set to generate same-sense mutations (SSMs) in the same open reading frame (ORF). Statistical analyses of the data from all 38 double additional base substitution-containing pegRNAs across nine target sites (FIG. 21D) showed that adding double additional base substitutions at positions 1/4, 2/5 and 3/6 could significantly enhance the intended single-base editing frequencies (median 1.20-, 1.90- and 1.41-fold, respectively, FIG. 21E).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for generating a target mutation to a protein in a cell, comprising introducing to the cell a prime editing system, wherein the prime editing system comprises a fusion protein comprising a nickase and a reverse transcriptase, and a prime editing guide RNA (pegRNA) encoding the target mutation and one or more silent or conservative mutations within 20 nucleotides from the target mutation.

2. The method of claim 1, wherein the pegRNA encodes at least 1 silent or conservative mutations.

3. The method of claim 1, wherein the pegRNA encodes from 2 to 4 silent or conservative mutations.

4. The method of claim 1, wherein each mutation is in a different codon from other mutations.

5. The method of claim 1, wherein the nickase is Cas9 H840A.

6. The method of claim 1, wherein the reverse transcriptase is M-MLV reverse transcriptase.

7. The method of claim 1, wherein the fusion protein is introduced as a recombinant DNA encoding the fusion protein.

8. The method of claim 1, wherein the pegRNA is introduced as a recombinant DNA encoding the pegRNA.

9. The method of claim 1, wherein the target mutation is at one or more residues of S19, Q24, D30, or K31 of human ACE2 (angiotensin-converting enzyme 2), wherein the positions are according to SEQ ID NO:1.

10. The method of claim 9, wherein the target mutation is at one or more residues of Q24, D30, K31A or K353 of human ACE2 (angiotensin-converting enzyme 2), wherein the positions are according to SEQ ID NO:1.

11. The method of claim 10, wherein the target mutation is a non-conservative mutation.

12. The method of claim 10, wherein the mutation comprises K353del, Q24A/D30A/K31A or Q24A/D30K/K31A.

13. A method for introducing mutations to a protein in a cell, comprising introducing to the cell a prime editing system, wherein the prime editing system comprises a fusion protein comprising a nickase and a reverse transcriptase, and a prime editing guide RNA (pegRNA) encoding two or more mutations within the coding sequence of the protein, wherein at least one of the mutations is a silent or conservative mutation and is within 20 nucleotides from another of mutations.

14-29. (canceled)

30. A method for conducting genetic editing in a cell at a target site, comprising introducing to the cell:
a first construct encoding a nickase, and
one or more second construct encoding (a) a prime editing guide RNA (pegRNA) capable of identifying the target site and including genetic information for editing the target site, (b) a single guide RNA (sgRNA) capable of directing the nickase to nick a non-edited DNA strand of the target site, wherein the pegRNA or the sgRNA includes a tag sequence, and (c) a reverse-transcriptase fused to an RNA recognition peptide capable of binding to the tag sequence.

31. The method of claim 30, wherein the first construct is packaged in a viral particle and the one or more second construct is packaged in a second viral particle.

32. The method of claim 30, wherein the viral particles are AAV particles.

33. The method of claim 30, wherein the sgRNA includes the tag sequence.

34. The method of claim 30, wherein the nickase is nCas(H840A).

35. The method of claim 30, wherein the tag sequence and the RNA recognition peptide, respectively, are selected from the group consisting of MS2/MS2 coat protein (MCP), PP7/PP7 coat protein (PCP), and boxB/boxB coat protein (N22p).
